(19) United States
(12) Patent Application Publication
(10) Pub. No.: US 2024/0242400 A1
(43) Pub. Date: Jul. 18, 2024

(54) SYSTEMS AND METHODS FOR MEDICAL IMAGING

(71) Applicant: SHANGHAI UNITED IMAGING HEALTHCARE CO., LTD., Shanghai (CN)

(72) Inventors: Xiaoyue GU, Shanghai (CN); Chao WANG, Shanghai (CN); Yang LYU, Shanghai (CN); Runxia MA, Shanghai (CN); Xiaochun XU, Shanghai (CN); Zijun JI, Shanghai (CN); Huayang LIU, Shanghai (CN); Hai WANG, Shanghai (CN); Chenhao XUE, Shanghai (CN)

(73) Assignee: SHANGHAI UNITED IMAGING HEALTHCARE CO., LTD., Shanghai (CN)

(21) Appl. No.: 18/434,934

(22) Filed: Feb. 7, 2024

Related U.S. Application Data
(63) Continuation of application No. PCT/CN2022/113544, filed on Aug. 19, 2022.

(30) Foreign Application Priority Data
Aug. 19, 2021 (CN) 202110952756.6
Oct. 20, 2021 (CN) 202111221748.0
Dec. 29, 2021 (CN) 202111634583.X

(51) Int. Cl. G06T 11/00; G06T 7/00

(52) U.S. Cl. CPC G06T 11/008; G06T 7/0014; G06T 2207/10081; G06T 2207/10088; G06T 2207/10104; G06T 2207/30004; G06T 2210/41; G06T 2211/448

(57) ABSTRACT

The present disclosure provides systems and methods for medical imaging. The system may obtain a scout image of a subject lying on a scanning table. The scanning table may include N portions corresponding to N bed positions of a target scan, and an i^{th} portion of the N portions may correspond to an i^{th} bed position of the N bed positions. For the i^{th} bed position, the system may determine one or more body parts of the subject located at the i^{th} portion of the scanning table based on the scout image; and determine at least one scanning parameter or reconstruction parameter corresponding to the i^{th} bed position based on the one or more body parts of the subject.

### CROSS-REFERENCE TO RELATED APPLICATIONS

This application is a continuation of International Application No. PCT/CN2022/113544, filed on Aug. 19, 2022, which claims priority to Chinese Patent application Ser. No. 20/211,0952756.6, filed on Aug. 19, 2021, Chinese Patent Application No. 202111221748.0, filed on Oct. 20, 2021, and Chinese Patent Application No. 202111634583.X, filed on Dec. 29, 2021, the contents of each of which are incorporated herein by reference.

### TECHNICAL FIELD

The present disclosure generally relates to imaging technology, and more particularly, relates to systems and methods for medical imaging.

### BACKGROUND

Medical imaging techniques (e.g., nuclear imaging) have been widely used in a variety of fields including, e.g., medical treatments and/or diagnosis. However, due to limitations including, e.g., a length of a detector in an axial direction, a field of view (FOV) of an imaging device (e.g., a PET device), etc., a medical scan needs to be performed by performing multiple sub-scans on a subject. For example, for performing a whole-body scan, a scanning table may be moved to different bed positions so that different body parts of the subject may be scanned in sequence. Conventionally, body part(s) of the subject scanned at a specific bed position need to be determined manually, and scanning parameters or reconstruction parameters of the specific bed position also need to be determined manually based on the determined body part(s). Further, after the medical scan is performed, the image quality of a resulting medical image needs to be manually evaluated by a user, which is time-consuming, labor-intensive, and inefficient.

Therefore, it is desirable to provide systems and methods for medical imaging, which can efficiently reduce the labor consumption and improve the efficiency of the scan preparation and image quality analysis.

### SUMMARY

In an aspect of the present disclosure, a method for medical imaging is provided. The method may be implemented on at least one computing device, each of which may include at least one processor and a storage device. The method may include obtaining a scout image of a subject lying on a scanning table, the scanning table including N portions corresponding to N bed positions of a target scan, and an i^{th} portion of the N portions corresponding to an i^{th} bed position of the N bed positions. For the i^{th} bed position, the method may include determining one or more body parts of the subject located at the i^{th} portion of the scanning table based on the scout image, and determining at least one scanning parameter or reconstruction parameter corresponding to the i^{th} bed position based on the one or more body parts of the subject.

In another aspect of the present disclosure, a system for medical imaging is provided. The system may include at least one storage device including a set of instructions, and at least one processor configured to communicate with the at least one storage device. When executing the set of instructions, the system may be configured to direct the system to perform the following operations. The system may obtain a scout image of a subject lying on a scanning table, the scanning table including N portions corresponding to N bed positions of a target scan, and an i^{th} portion of the N portions corresponding to an i^{th} bed position of the N bed positions. For the i^{th} bed position, the system may determine one or more body parts of the subject located at the i^{th} portion of the scanning table based on the scout image, and determine at least one scanning parameter or reconstruction parameter corresponding to the i^{th} bed position based on the one or more body parts of the subject.

In still another aspect of the present disclosure, a non-transitory computer-readable medium storing at least one set of instructions is provided. When executed by at least one processor, the at least one set of instructions may direct the at least one processor to perform a method. The method may include obtaining a scout image of a subject lying on a scanning table, the scanning table including N portions corresponding to N bed positions of a target scan, and an i^{th} portion of the N portions corresponding to an i^{th} bed position of the N bed positions. For the i^{th} bed position, the method may include determining one or more body parts of the subject located at the i^{th} portion of the scanning table based on the scout image, and determining at least one scanning parameter or reconstruction parameter corresponding to the i^{th} bed position based on the one or more body parts of the subject.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a block diagram illustrating an exemplary processing device according to some embodiments of the present disclosure;

FIG. 3 is a flowchart illustrating an exemplary process for obtaining a target image according to some embodiments of the present disclosure;

FIG. 4 is a flowchart illustrating an exemplary process for determining one or more body parts of a subject located at an i^{th} portion of a scanning table according to some embodiments of the present disclosure;

FIG. 5A is a schematic diagram illustrating an exemplary scout image including image portions corresponding to a plurality of bed positions according to some embodiments of the present disclosure;

FIG. 5B is a schematic diagram illustrating another exemplary scout image including image portions corresponding to a plurality of bed positions according to some embodiments of the present disclosure;

FIG. 5C is a schematic diagram illustrating an exemplary scout image including feature points according to some embodiments of the present disclosure;

FIG. 6 is a schematic diagram illustrating an exemplary user interface according to some embodiments of the present disclosure;

FIG. 7 is a flowchart illustrating an exemplary process for determining whether a first image includes image artifacts according to some embodiments of the present disclosure;

FIG. 8 is a flowchart illustrating an exemplary process for determining one or more target image blocks according to some embodiments of the present disclosure;

FIG. 9 is a schematic diagram illustrating an exemplary process for determining one or more target image blocks according to some embodiments of the present disclosure;

FIG. 10 is a schematic diagram illustrating an exemplary process for determining one or more target image blocks according to some embodiments of the present disclosure;

FIG. 11 is a schematic diagram illustrating an exemplary process for generating a corrected first image according to some embodiments of the present disclosure;

FIG. 12 is a schematic diagram illustrating an exemplary process for training an initial model according to some embodiments of the present disclosure;

FIG. 13 is a flowchart illustrating an exemplary process for determining whether a target scan needs to be re-performed according to some embodiments of the present disclosure;

FIG. 14 is a flowchart illustrating an exemplary process for determining whether a target scan needs to be re-performed according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, the term “image” may refer to a two-dimensional (2D) image, a three-dimensional (3D) image, or a four-dimensional (4D) image (e.g., a time series of 3D images). In some embodiments, the term “image” may refer to an image of a region (e.g., a region of interest (ROI)) of a subject. In some embodiment, the image may be a medical image, an optical image, etc.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

The present disclosure relates to systems and methods for medical imaging. The method may include obtaining a scout image of a subject lying on a scanning table. The scanning table may include N portions corresponding to N bed positions of a target scan, and an i^{th} portion of the N portions corresponding to an i^{th} bed position of the N bed positions. A plurality of feature points of the subject may be identified from the scout image. According to a corresponding relationship between the plurality of feature points and a plurality of body part classifications and a positional relationship between the plurality of feature points and the i^{th} portion of the scanning table, one or more body parts of the subject located at the i^{th} portion of the scanning table may be determined automatically for the i^{th} bed position, and at least one scanning parameter or reconstruction parameter corresponding to the i^{th} bed position may be determined automatically based on the one or more body parts of the subject corresponding to the i^{th} bed position, which may reduce time and/or labor consumption, and improve the efficiency of parameter determination.

In addition, a target image (also referred to as a first image) of the subject may be captured by the target scan based on the at least one scanning parameter or reconstruction parameter. The method may include determining whether the target image includes image artifacts and/or the target scan needs to be re-performed, automatically, which may improve an image quality of the target image, and further reduce the time and/or labor consumption and improve the user experience.

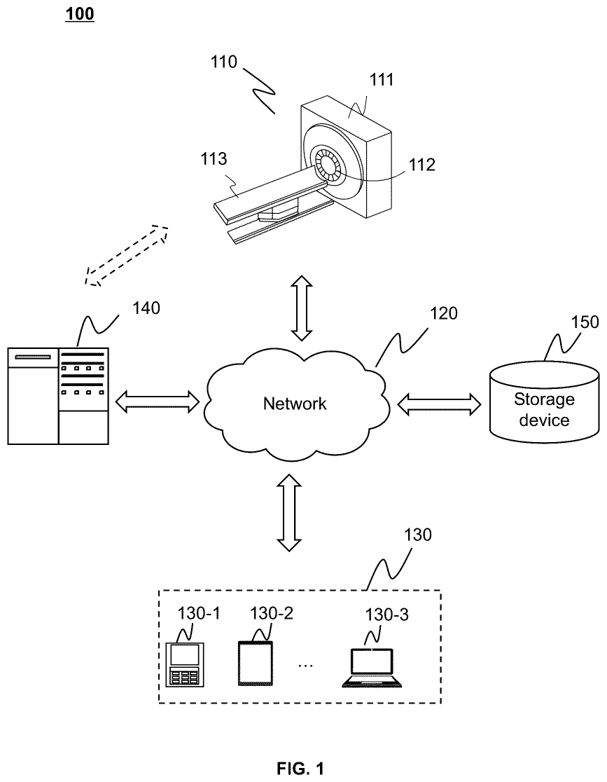

FIG. 1 is a schematic diagram illustrating an exemplary imaging system 100 according to some embodiments of the present disclosure. As shown in FIG. 1, the imaging system 100 may include an imaging device 110, a network 120, one or more terminals 130, a processing device 140, and a storage device 150. In some embodiments, the imaging device 110, the processing device 140, the storage device 150, and/or the terminal(s) 130 may be connected to and/or communicate with each other via a wireless connection (e.g., the network 120), a wired connection, or a combination thereof. The connection between the components in the imaging system 100 may be variable. Merely by way of example, the imaging device 110 may be connected to the processing device 140 through the network 120, as illustrated in FIG. 1. As another example, the imaging device 110 may be connected to the processing device 140 directly. As a further example, the storage device 150 may be connected to the processing device 140 through the network 120, as illustrated in FIG. 1, or connected to the processing device 140 directly.

The imaging device 110 may be configured to generate or provide image data by scanning a subject or at least a part of the subject. For example, the imaging device 110 may obtain the image data of the object by performing a scan (e.g., a target scan, a reference scan, etc.) on the subject. In some embodiments, the imaging device 110 may include a single modality imaging device. For example, the imaging device 110 may include a positron emission tomography (PET) device, a single-photon emission computed tomography (SPECT) device, a computed tomography (CT) device, a magnetic resonance (MR) device, or the like. In some embodiments, the imaging device 110 may include a multi-modality imaging device. Exemplary multi-modality imaging devices may include a positron emission tomography-computed tomography (PET-CT) device, a positron emission tomography-magnetic resonance imaging (PET-MRI) device, a single-photon emission computed tomography-computed tomography (SPECT-CT) device, etc. The multi-modality scanner may perform multi-modality imaging simultaneously or in sequence. For example, the PET-CT device may generate structural X-ray CT image data and functional PET image data simultaneously or in sequence. The PET-MRI device may generate MRI data and PET data simultaneously or in sequence.

The subject may include patients or other experimental subjects (e.g., experimental mice or other animals). In some embodiments, the subject may be a patient or a specific portion, organ, and/or tissue of the patient. For example, the subject may include the head, the neck, the thorax, the heart, the stomach, a blood vessel, soft tissue, a tumor, nodules, or the like, or any combination thereof. In some embodiments, the subject may be non-biological. For example, the subject may include a phantom, a man-made object, etc.

Merely by way of example, the imaging device 110 may include a PET device. The PET device may include a gantry 111, a detector 112, a scanning table 113, etc. The subject may be placed on the scanning table 113 and transmitted to a detection region of the imaging device 110 for scanning (e.g., a PET scan). In some embodiments, the scanning table 113 may include a plurality of position codes indicating different positions along a long axis of the scanning table 113. For example, at a specific position at the scanning table 113, a distance from the specific position to a front end or a rear end of the scanning table 113 may be marked as a position code. The front end of the scanning table 113 refers to an end close to the imaging device 110. The rear end of the scanning table 113 refers to an end away from the imaging device 110.

To prepare for a PET scan, a radionuclide (also referred to as “PET tracer” or “PET tracer molecules”) may be introduced into the subject. Substances (e.g., glucose, protein, nucleic acid, fatty acid, etc.) necessary for the metabolism of the subject may be labelled with the radionuclide. The radionuclide may aggregate, with the circulation and metabolism of the subject, in a certain region, for example, cancer lesions, myocardial abnormal tissue, etc. The PET tracer may emit positrons in the detection region when it decays. An annihilation (also referred to as “annihilation event” or “coincidence event”) may occur when a positron collides with an electron. The annihilation may produce two gamma photons, which may travel in opposite directions. The line connecting the detector units that detecting the two gamma photons may be defined as a “line of response (LOR).” The detector 112 set on the gantry 111 may detect the annihilation events (e.g., gamma photons) emitted from the detection region.

The annihilation events emitted from the detection region may be used to generate PET data (also referred to as the image data). In some embodiments, the detector 112 used in the PET scan may include crystal elements and photomultiplier tubes (PMT).

In some embodiments, the PET scan may be divided into a plurality of sub-scans due to limitations including, e.g., a length of the detector 112 of the imaging device 110 along an axial direction, a field of view (FOV) of the imaging device 110, etc. For example, a whole-body PET scan may be performed by dividing the PET scan into a plurality of sub-scans based on a length of the FOV of the imaging device. The scanning table 113 may be positioned at different bed positions to perform the sub-scans. Merely by way of example, the scanning table 113 may be positioned at a first bed position to perform a first sub-scan, then the scanning table 113 may be moved to a second bed position to perform a second sub-scan. When the scanning table 113 is at the first bed position, a first portion of the scanning table 113 is within the FOV of the imaging device so that a portion of the subject (e.g., the head) on the first portion may be scanned by the first sub-scan. When the scanning table 113 is at the second bed position, a second portion of the scanning table 113 is within the FOV of the imaging device so that a portion of the subject on the second portion (e.g., the chest) may be scanned by the second sub-scan. In other words, each of the plurality of sub-scans may correspond to a distinctive bed position, and each bed position may correspond to a portion of the scanning table 113. A portion of the scanning table 113 corresponding to a specific bed position refers to a portion within the FOV of the imaging device 110 when the scanning table is at the specific bed position.

In some embodiments, the scanning table 113 may include a plurality of portions, and each of the plurality of portions may correspond to a bed position of the PET scan. For example, the scanning table 113 may include N portions corresponding to N bed positions of the PET scan, and an i^{th} portion of the N portions may correspond to an i^{th} bed position of the N bed positions. Merely by way of example, the scanning table 113 is 2 meters, and the length of the FOV of the imaging device along the long axis is 400 millimeters, the scanning table 113 may include five portions corresponding to five bed positions, wherein a first bed position may correspond to a first portion of the scanning table 113 within a range from 0 millimeters to 400 millimeters, a second bed position may correspond to a second portion of the scanning table 113 within a range from 400 millimeters to 800 millimeters, a third bed position may correspond to a third portion of the scanning table 113 within a range from 800 millimeters to 1200 millimeters, a fourth bed position may correspond to a fourth portion of the scanning table 113 within a range from 1200 millimeters to 1600 millimeters, and a fifth bed position may correspond to a fifth portion of the scanning table 113 within a range from 1600 millimeters to 2000 millimeters.

In some embodiments, two portions of the scanning table 113 corresponding to adjacent bed positions may include no overlapping region. That is, no portion of the subject may be scanned twice during the PET scan. In some embodiments, two portions of the scanning table 113 corresponding to adjacent bed positions may include an overlapping region. That is, a portion of the subject may be scanned twice during the PET scan. For example, if the first bed position corresponds to the first portion of the scanning table 113 within a range from 0 millimeters to 400 millimeters, and the second bed position corresponds to the second portion of the scanning table 113 within a range from 360 millimeters to 760 millimeters, a portion within a range from 360 millimeters to 400 millimeters may be the overlapping region.

The network 120 may include any suitable network that can facilitate the exchange of information and/or data for the imaging system 100. In some embodiments, one or more components (e.g., the imaging device 110, the terminal 130, the processing device 140, the storage device 150, etc.) of the imaging system 100 may communicate information and/or data with one or more other components of the imaging system 100 via the network 120. For example, the processing device 140 may obtain image data from the imaging device 110 via the network 120. As another example, the processing device 140 may obtain user instructions from the terminal 130 via the network 120. In some embodiments, the network 120 may include one or more network access points.

The terminal(s) 130 may include a mobile device 130-1, a tablet computer 130-2, a laptop computer 130-3, or the like, or any combination thereof. In some embodiments, the mobile device 130-1 may include a smart home device, a wearable device, a mobile device, a virtual reality device, an augmented reality device, or the like, or any combination thereof. In some embodiments, the terminal(s) 130 may be part of the processing device 140.

The processing device 140 may process data and/or information obtained from one or more components (the imaging device 110, the terminal(s) 130, and/or the storage device 150) of the imaging system 100. For example, for each bed position of the scanning table 113, the processing device 140 may determine one or more body parts of the subject located at the corresponding portion of the scanning table 113, and determine at least one scanning parameter or reconstruction parameter corresponding to the bed position based on the one or more body parts of the subject. As another example, the processing device 140 may obtain a target image (e.g., a first image) of the subject captured by a target scan, and determine whether the target image includes image artifacts. As still another example, the processing device 140 may determine whether the target scan needs to be re-performed based on one or more quality parameters of the target image. In some embodiments, the processing device 140 may be a single server or a server group. The server group may be centralized or distributed. In some embodiments, the processing device 140 may be local or remote. In some embodiments, the processing device 140 may be implemented on a cloud platform.

In some embodiments, the processing device 140 may be implemented by a computing device. For example, the computing device may include a processor, a storage, an input/output (I/O), and a communication port. The processor may execute computer instructions (e.g., program codes) and perform functions of the processing device 140 in accordance with the techniques described herein. The computer instructions may include, for example, routines, programs, objects, components, data structures, procedures, modules, and functions, which perform particular functions described herein. In some embodiments, the processing device 140, or a portion of the processing device 140 may be implemented by a portion of the terminal 130.

The storage device 150 may store data/information obtained from the imaging device 110, the terminal(s) 130, and/or any other component of the imaging system 100. In some embodiments, the storage device 150 may include a mass storage, a removable storage, a volatile read-and-write memory, a read-only memory (ROM), or the like, or any combination thereof. In some embodiments, the storage device 150 may store one or more programs and/or instructions to perform exemplary methods described in the present disclosure.

In some embodiments, the storage device 150 may be connected to the network 120 to communicate with one or more other components in the imaging system 100 (e.g., the processing device 140, the terminal(s) 130, etc.). One or more components in the imaging system 100 may access the data or instructions stored in the storage device 150 via the network 120. In some embodiments, the storage device 150 may be directly connected to or communicate with one or more other components in the imaging system 100 (e.g., the processing device 140, the terminal(s) 130, etc.). In some embodiments, the storage device 150 may be part of the processing device 140.

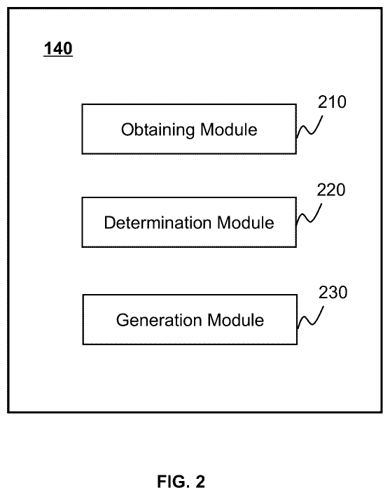

FIG. 2 is a block diagram illustrating an exemplary processing device 140 according to some embodiments of the present disclosure. In some embodiments, the modules illustrated in FIG. 2 may be implemented on the processing device 140. In some embodiments, the processing device 140 may be in communication with a computer-readable storage medium (e.g., the storage device 150 illustrated in FIG. 1) and may execute instructions stored in the computer-readable storage medium. The processing device 140 may include an obtaining module 210, a determination module 220, and a generation module 230.

The obtaining module 210 may be configured to obtain a scout image of a subject lying on a scanning table. The scout image may refer to an image for determining information used to guide the implementation of a target scan. More descriptions regarding the obtaining of the scout image of the subject may be found elsewhere in the present disclosure. See, e.g., operation 302 and relevant descriptions thereof.

The determination module 220 may be configured to determine, based on the scout image, one or more body parts of the subject located at an i^{th} portion of the scanning table for an i^{th} bed position. In some embodiments, each of N bed positions may correspond to one or more body parts of the subject located at the corresponding portion of the scanning table. More descriptions regarding the determination of the one or more body parts of the subject located at the i^{th} portion of the scanning table for the i^{th} bed position may be found elsewhere in the present disclosure. See, e.g., operation 304 and relevant descriptions thereof.

The generation module 230 may be configured to determine at least one scanning parameter or reconstruction parameter corresponding to the i^{th} bed position based on the one or more body parts of the subject for the i^{th} bed position. The at least one scanning parameter corresponding to the i^{th} bed position may be used in the i^{th} sub-scan of the target scan (i.e., a sub-scan performed when the scanning table is at the i^{th} bed position). More descriptions regarding the determination of the at least one scanning parameter or reconstruction parameter may be found elsewhere in the present disclosure. See, e.g., operation 306 and relevant descriptions thereof.

In some embodiments, the obtaining module 210 may further be configured to obtain the target image captured by the target scan. More descriptions regarding the obtaining of the target image may be found elsewhere in the present disclosure. See, e.g., operation 308 and relevant descriptions thereof.

In some embodiments, the target image may be further processed. For example, the determination module 220 may perform artifact analysis on the target image, for example, determine whether the target image includes image artifacts. As another example, the determination module 220 may determine whether the target scan needs to be re-performed by analyzing the target image.

It should be noted that the above descriptions of the processing device 140 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the processing device 140 may include one or more other modules. For example, the processing device 140 may include a storage module to store data generated by the modules in the processing device 140. In some embodiments, any two of the modules may be combined as a single module, and any one of the modules may be divided into two or more units. For example, the obtaining module 210 may include a first obtaining unit for obtaining the scout image and a second obtaining unit for obtaining the target image. As another example, the determination module 220 may include a first determination unit, a second determination unit, and a third determination unit, wherein the first determination unit may determine, based on the scout image, the one or more body parts of the subject located at the i^{th} portion of the scanning table for the i^{th} bed position, the second determination unit may perform artifact analysis on the target image, and the third determination unit may determine whether the target scan needs to be re-performed by analyzing the target image.

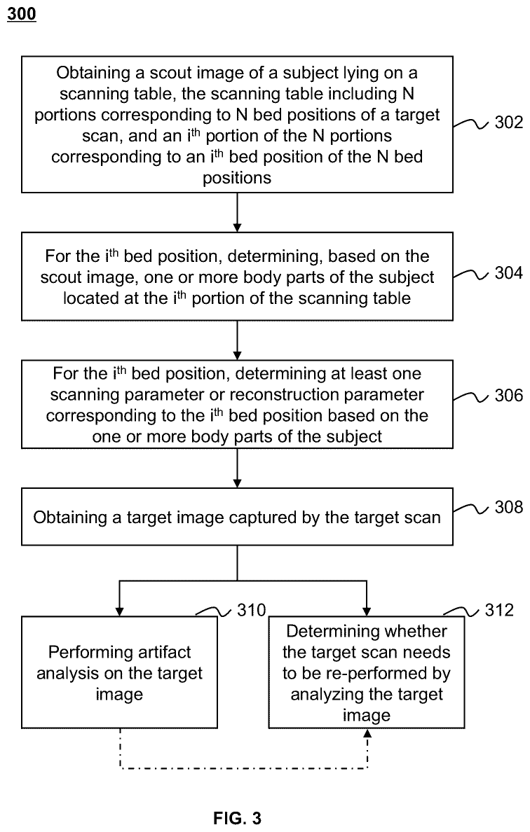

FIG. 3 is a flowchart illustrating an exemplary process for obtaining a target image according to some embodiments of the present disclosure. Process 300 may be implemented in the imaging system 100 illustrated in FIG. 1. For example, the process 300 may be stored in the storage device 150 in the form of instructions (e.g., an application), and invoked and/or executed by the processing device 140. The operations of the illustrated process presented below are intended to be illustrative. In some embodiments, the process 300 may be accomplished with one or more additional operations not described, and/or without one or more of the operations discussed. Additionally, the order in which the operations of the process 300 as illustrated in FIG. 3 and described below is not intended to be limiting.

In some embodiments, the target image (or referred to as a first image) may be obtained by performing a target scan of a subject using a first imaging device, and the subject may lie on a scanning table during the target scan. For example, the target scan may be a PET scan to obtain a PET image of the subject. In some embodiments, the target scan may include N sub-scans, and the scanning table may include N portions corresponding to N bed positions of the target scan. During the target scan, the scanning table may be moved to the N bed positions, respectively, for performing the N sub-scans. For example, for performing an i^{th} sub-scan, the scanning table is moved an i^{th} bed position, an i^{th} portion of the scanning table may be placed within the FOV of the first imaging device, so that body part(s) lying on the i^{th} portion of the scanning table may be scanned. N may be a positive integer, and i^{th} may be a positive integer within a range from 1 to N.

Conventionally, body part(s) of the subject that are scanned in different sub-scans need to be determined manually. For example, a user needs to manually inspect the subject lying on the scanning table to determine which parts of the subject are scanned when the scanning table is located at different bed positions. Further, different scanning parameters or reconstruction parameters need to be manually determined for different body parts, which is time-consuming, labor-intensive, and inefficient. In order to reduce time and/or labor consumption and improve the efficiency of parameter determination, the process 300 may be performed.

In 302, the processing device 140 (e.g., the obtaining module 210) may obtain a scout image of the subject lying on the scanning table.

The scout image may refer to an image for determining information used to guide the implementation of the target scan. For example, before the target scan is performed on the subject, the processing device 140 may obtain the scout image of the subject to determine one or more body parts for each of the N bed positions of the target scan. For each bed position, the processing device 140 may further determine scanning parameter(s) and/or reconstruction parameter(s) based on the corresponding body part(s). As another example, the processing device 140 may determine the position of the head of the subject based on the scout image. Therefore, the head of the subject may be scanned during the target scan based on the determined position, while no other parts may be scanned, thereby improving the efficiency of the target scan.

In some embodiments, the processing device 140 may cause a second imaging device to perform a positioning scan (i.e., a pre-scan) to obtain the scout image of the subject. The second imaging device may be the same as or different from the first imaging device for performing the target scan. Merely by way of example, the first imaging device may be a PET scanner, and the second imaging device may be a CT scanner. Optionally, the PET scanner and the CT scanner may be integrated into a PET/CT scanner. In some embodiments, the scout image may include one or more plane images obtained by performing plain scan(s) (or referred to as fast scan(s)) on the subject using the CT scanner. Exemplary plane images may include an anteroposterior image and a lateral image. In some embodiments, the subject may be asked to hold the same posture during the scout scan and the target scan.

In 304, for the i^{th} bed position, the processing device 140 (e.g., the determination module 220) may determine, based on the scout image, one or more body parts of the subject located at the i^{th} portion of the scanning table.

In some embodiments, each of the N bed positions may correspond to one or more body parts of the subject located at the corresponding portion of the scanning table.

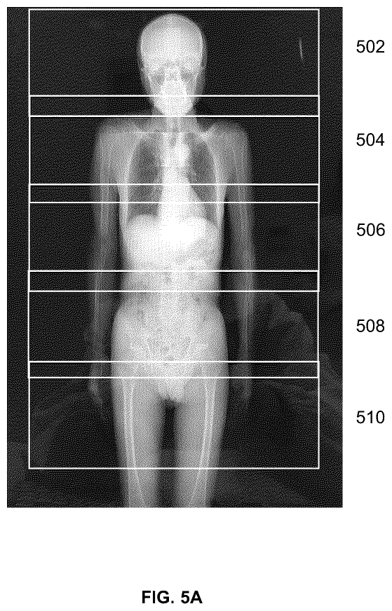

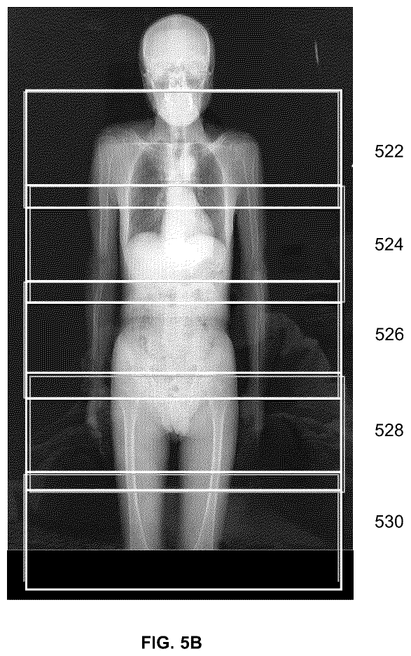

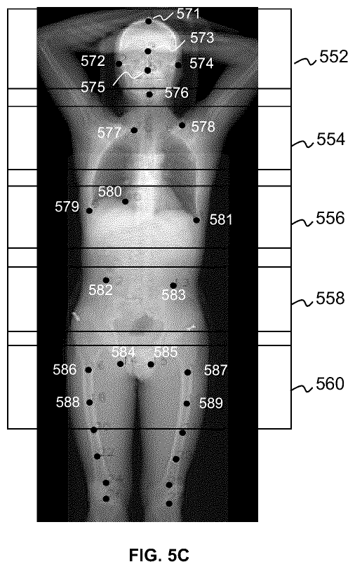

Merely by way of example, referring to FIG. 5A, FIG. 5A is a schematic diagram illustrating an exemplary scout image including image portions corresponding to a plurality of bed positions according to some embodiments of the present disclosure. As shown in FIG. 5A, an image portion 502 including a representation of the head may correspond to a first bed position, an image portion 504 including a representation of the chest may correspond to a second bed position, an image portion 506 including a representation of the abdomen may correspond to a third bed position, an image portion 508 including a representation of the pelvis may correspond to a fourth bed position, and an image portion 510 including a representation of the lower extremities may correspond to a fifth bed position. Correspondingly, the first bed position may correspond to the head (i.e., the head of the patient may be scanned in the target scan when the scanning table is located at the first bed position), the second bed position may correspond to the chest, the third bed position may correspond to the abdomen, the fourth bed position may correspond to the pelvis, and the fifth bed position may correspond to the lower extremities.

As another example, referring to FIG. 5B, FIG. 5B is a schematic diagram illustrating another exemplary scout image including image portions corresponding to a plurality of bed positions according to some embodiments of the present disclosure. As shown in FIG. 5B, an image portion 522 including a representation of the chest may correspond to a first bed position, an image portion 524 including a representation of the abdomen may correspond to a second bed position, an image portion 526 including a representation of the pelvis may correspond to a third bed position, an image portion 528 including a representation of the lower extremities may correspond to a fourth bed position, and an image portion 530 including a representation of the lower extremities may correspond to a fifth bed position. Correspondingly, the first bed position may correspond to the chest, the second bed position may correspond to the abdomen, the third bed position may correspond to the pelvis, and the fourth bed position and the fifth bed position may correspond to the lower extremities.

In some embodiments, the processing device 140 may identify a plurality of feature points of the subject from the scout image. A feature point may refer to a landmark point that belongs to a specific body part of the subject and can be used to identify different body parts of the subject from the scout image. Exemplary feature points may include the calvaria, the zygomatic bone, the mandible, the shoulder joint, the apex of the lung, the diaphragmatic dome, the femoral joint, the knee, or the like, or any combination thereof. For example, the processing device 140 may determine a morphological structure (e.g., positions of the bones and organs) of the scanned object based on the scout image, and further identify feature points of the subject based on the morphological structure. As another example, the processing device 140 may identify feature points of the subject from the scout image based on a recognition model. For instance, the processing device 140 may input the scout image of the subject to the recognition model, and the recognition model may output information (e.g., position information) relating to the feature points of the subject. In some embodiments, the recognition model may include a neural network model, a logistic regression model, a support vector machine, etc.

In some embodiments, the recognition model may be trained based on a plurality of first training samples with labels. Each of the plurality of first training samples may be a sample scout image of a sample subject, and the corresponding label may include one or more feature points marked in the sample scout image. In some embodiments, the labels of the first training samples may be added by manual labeling or other manners. By using the recognition model, the accuracy and efficiency of the identification of feature points may be improved.

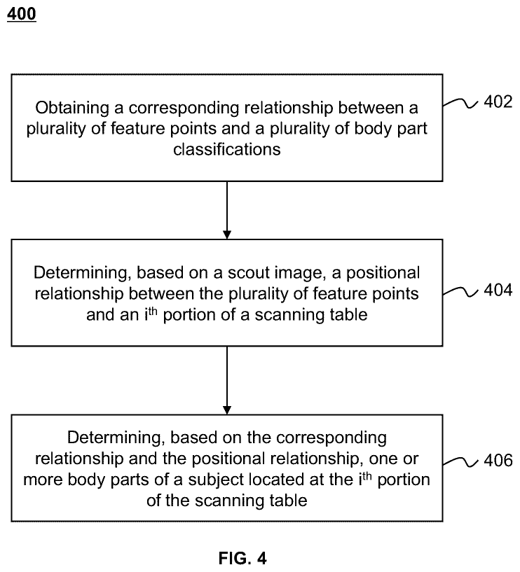

Further, for the i^{th} bed position, the processing device 140 may determine, based on feature points, the one or more body parts of the subject located at the i^{th} portion of the scanning table. For example, the processing device 140 may obtain a corresponding relationship (also referred to as a first corresponding relationship) between feature points and a plurality of body part classifications, and determine a positional relationship between feature points and the i^{th} portion of the scanning table based on the scout image. Further, the processing device 140 may determine the one or more body parts of the subject located at the i^{th} portion of the scanning table based on the corresponding relationship and the positional relationship. More descriptions regarding the determination of the one or more body parts located at the i^{th} portion of the scanning table may be found in elsewhere in the present disclosure (e.g., FIGS. 4 and 5C, and the descriptions thereof).

In 306, for the i^{th} bed position, the processing device 140 (e.g., the determination module 220) may determine at least one scanning parameter or reconstruction parameter corresponding to the i^{th} bed position based on the one or more body parts of the subject.

The at least one scanning parameter corresponding to the i^{th} bed position may be used in the i^{th} sub-scan of the target scan (i.e., a sub-scan performed when the scanning table is at the i^{th} bed position). Exemplary scanning parameters may include a scanning region, a scanning resolution, a scanning speed, or the like, or any combination thereof.

The at least one reconstruction parameter corresponding to the i^{th} bed position may be used to perform image reconstruction on image data captured by the i^{th} sub-scan of the target scan. Exemplary reconstruction parameters may include a reconstruction algorithm, a reconstruction speed, a reconstruction quality, a correction parameter, a slice thickness, or the like, or any combination thereof.

In some embodiments, different body parts of the subject may correspond to different scanning parameters. For example, scanning parameter(s) corresponding to the head of the subject may be different from scanning parameter(s) corresponding to the chest of the subject. Similarly, different body parts of the subject may correspond to different reconstruction parameters. In some embodiments, the scanning parameters and/or reconstruction parameters corresponding to each body part may be determined based on system default setting or set manually by a user (e.g., a technician, a doctor, a physicist, etc.).

In some embodiments, for the i^{th} bed position, the processing device 140 may determine the corresponding scanning parameter(s) and/or reconstruction parameters(s) based on the one or more body parts of the subject corresponding to the i^{th} bed position. Merely by way of example, referring to FIG. 5A, the processing device 140 may determine scanning parameter(s) and/or reconstruction parameters(s) corresponding to the first bed position based on the head, determine scanning parameter(s) and/or reconstruction parameters(s) corresponding to the second bed position based on the chest, determine scanning parameter(s) and/or reconstruction parameters(s) corresponding to the third bed position based on the abdomen, determine scanning parameter(s) and/or reconstruction parameters(s) corresponding to the fourth bed position based on the pelvis, and determine scanning parameter(s) and/or reconstruction parameters(s) corresponding to the fifth bed position based on the lower extremities. Referring to FIG. 5B, the processing device 140 may determine scanning parameter(s) and/or reconstruction parameters(s) corresponding to the first bed position based on the chest, determine scanning parameter(s) and/or reconstruction parameters(s) corresponding to the second bed position based on the abdomen, determine scanning parameter(s) and/or reconstruction parameters(s) corresponding to the third bed position based on the pelvis, and determine scanning parameter(s) and/or reconstruction parameters(s) corresponding to the fourth bed position and the fifth bed position based on the lower extremities.

In some embodiments, a second corresponding relationship, which records different body parts and their corresponding scanning parameter(s) and/or reconstruction parameters(s), may be stored in a storage device (e.g., the storage device 150). For the i^{th} bed positions, the processing device 140 may obtain the corresponding scanning parameter(s) and/or reconstruction parameters(s) based on the second corresponding relationship and the one or more body parts of the subject corresponding to the i^{th} bed position. For example, the scanning parameter(s) corresponding to the head and the scanning parameter(s) corresponding to the chest may be stored in a look-up table in the storage device 150. If the first bed position corresponds to the head, the processing device 140 may obtain the scanning parameter(s) corresponding to the head by looking up the look-up table.

In some embodiments, the determined scanning parameter(s) or reconstruction parameter(s) may be further checked manually. For example, the determined scanning parameter(s) or reconstruction parameter(s) may be displayed on a user interface. The user may input an instruction (e.g., a selection instruction, a modification instruction, an acceptance instruction, etc.) in response to the displayed scanning parameter(s) or reconstruction parameter(s). Further, the processing device 140 may perform the target scan or the image reconstruction based on the instruction.

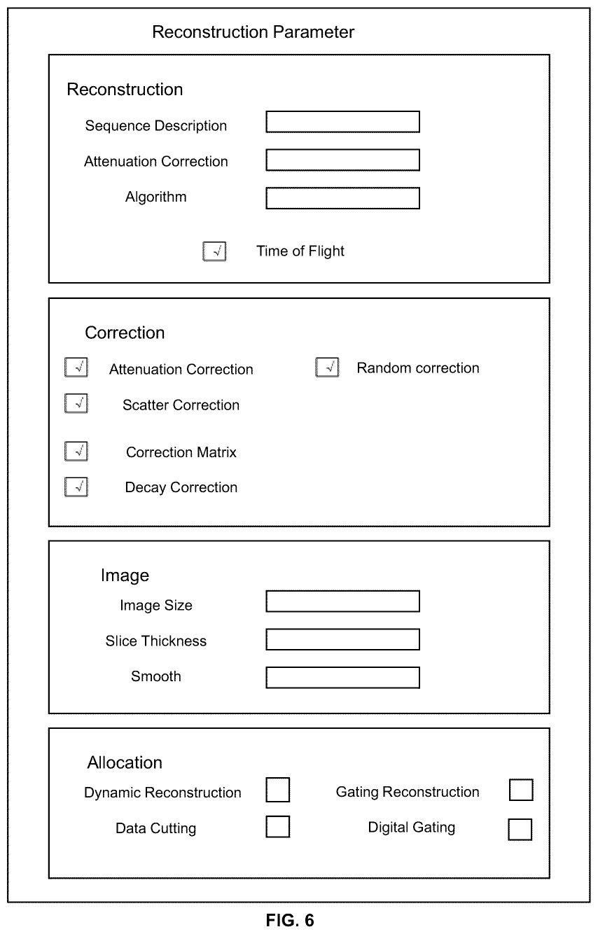

Merely by way of example, referring to FIG. 6, FIG. 6 is a schematic diagram illustrating an exemplary user interface according to some embodiments of the present disclosure. The user interface may be displayed to a user for manually checking the scanning parameters or reconstruction parameters.

As shown in FIG. 6, the user interface may include a list of reconstruction parameters. The list of reconstruction parameters may be divided into a reconstruction section, a correction section, an image section, and an allocation section. Each section may include multiple options. For example, the reconstruction section may include an option of sequence description, an option of attenuation correction, an option of algorithm, an option of time of flight, etc. As another example, the correction section may include an option of attenuation correction, an option of scatter correction, an option of correction matrix, an option of decay correction, an option of random correction, etc. As still another example, the image section may include an option of image size, an option of slice thickness, an option of smooth, etc. As a further example, the allocation section may include an option of dynamic reconstruction, an option of gating reconstruction, an option of data cutting, an option of digital gating, etc. Via the user interface, the user may confirm and/or modify the scanning parameter(s) and/or the reconstruction parameter(s) determined based on the body part(s) of the subject. By using the user interface to check the scanning parameter(s) and/or the reconstruction parameter(s), the interactivity of the scan may be improved.

In some embodiments, the one or more body parts of the subject located at the i^{th} portion of the scanning table may include a body part having physiological motion. Correspondingly, the at least one scanning parameter may include a motion detection parameter, and/or the at least one reconstruction parameter may include a motion correction parameter. In some embodiments, the motion detection parameter and/or the motion correction parameter may be used to reduce or eliminate the effect of the physiological motion on the target image (e.g., reducing image artifacts). Merely by way of example, a motion detection parameter may be determined to direct a monitoring device to collect a physiological signal (e.g., a respiratory signal and/or a cardiac signal) of the subject during the target scan. If the physiological signal indicates that a physiological motion (e.g., a respiratory motion, a heartbeat motion, etc.) of the subject is violent during the target scan, the processing device 140 may perform motion correction on the image data collected in the target scan to avoid image artifacts.

In some embodiments, the motion correction parameter may be used to correct a rigid motion of the body of the subject. For example, during the target scan, a rigid motion of a portion of the body parts (e.g., the head, the chest, the abdomen, etc.) may occur, and the processing device 140 may perform rigid motion correction on the image data collected in the target scan based on the motion correction parameter.

In some embodiments, the processing device 140 may monitor the movement of the subject during the target scan. For example, since the scout image has real-time interactivity, the processing device 140 may determine whether the one or more body parts of the subject still correspond to the i^{th} bed position during the target scan based on the scout image. As another example, the processing device 140 may receive a user instruction for determining whether the one or more body parts of the subject still correspond to the i^{th} bed position during the target scan. As still another example, the processing device 140 may continuously obtain images (e.g., optical images) of the subject to determine whether the one or more body parts of the subject still correspond to the i^{th} bed position during the target scan. In response to determining that the one or more body parts of the subject don't correspond to the i^{th} bed position, the processing device 140 may update one or more body parts of the subject corresponding to the i^{th} bed position, and update the at least one scanning parameter or reconstruction parameter corresponding to the i^{th} bed position. For instance, assuming that the subject is moved from a position as shown in FIG. 5A to a position as shown in FIG. 5B, the one or more body parts corresponding to the first bed position may be adjusted from the head to the chest.

In 308, the processing device 140 (e.g., the obtaining module 310) may obtain the target image captured by the target scan.

After the scanning parameter(s) and/or reconstruction parameter(s) are determined, the processing device 140 may obtain the target image by performing the target scan on the subject using the first imaging device based on the scanning parameter(s), and performing the image reconstruction on the image data based on the reconstruction parameter(s). For example, the i^{th} sub-scan of the target scan may be performed based on the scanning parameter(s) corresponding to the i^{th} bed position. In some embodiments, the first imaging device may be a PET device with a short axial FOV (e.g., a length of the FOV along the axial direction being shorter than a threshold). The i^{th} sub-scans corresponding to the N bed positions may be performed successively. When the i^{th} sub-scan is completed, the PET device may be adjusted to the at least one scanning parameter or reconstruction parameter corresponding to the (i+1)^{th} bed position for performing the (i+1)^{th} sub-scan.

The image reconstruction of the image data collected in the i^{th} sub-scan may be performed based on the reconstruction parameter(s) corresponding to the i^{th} bed position. In some embodiments, an image corresponding to each sub-scan may be generated, therefore, a plurality of images may be obtained. The images may be further stitched to generate the target image. In some embodiments, if the scanning parameter(s) corresponding to the i^{th} bed position are not determined in operation 306, the i^{th} sub-scan may be performed according to default scanning parameters or manually set scanning parameters. If the reconstruction parameter(s) corresponding to the i^{th} bed position are not determined in operation 306, the image reconstruction of the i^{th} sub-scan may be performed according to default reconstruction parameters or manually set reconstruction parameters.

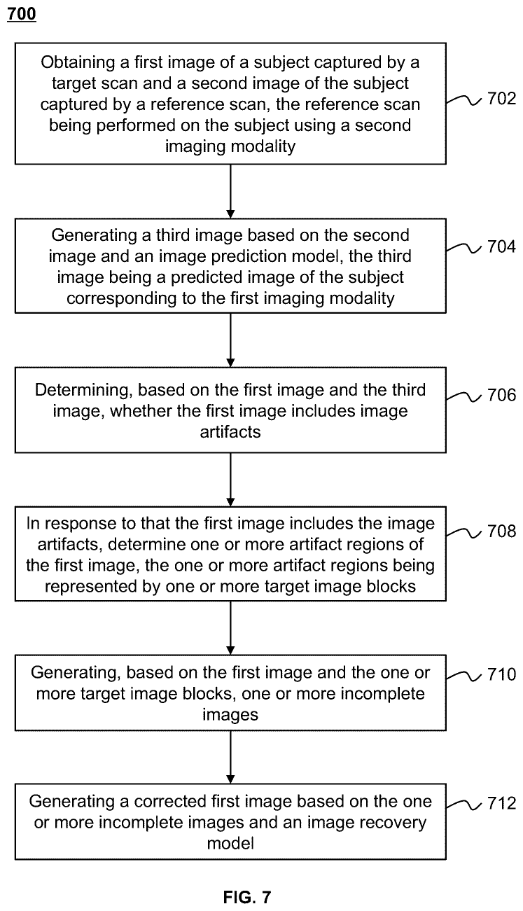

In some embodiments, the processing device 140 may further process the target image. For example, the processing device 140 may proceed to operation 310. In 310, the processing device 140 may further perform artifact analysis on the target image, for example, determine whether the target image includes image artifacts. For instance, the processing device 140 may obtain a second image of the subject captured by a reference scan. The target scan may be performed on the subject using a first imaging modality (e.g., PET), and the reference scan may be performed on the subject using a second imaging modality (e.g., CT or MRI). The processing device 140 may determine whether the target image includes image artifacts based on the target image (i.e., the first image) and the second image. More descriptions regarding the determination of whether the target image includes image artifacts may be found in elsewhere in the present disclosure (e.g., FIGS. 7-12 and the descriptions thereof).

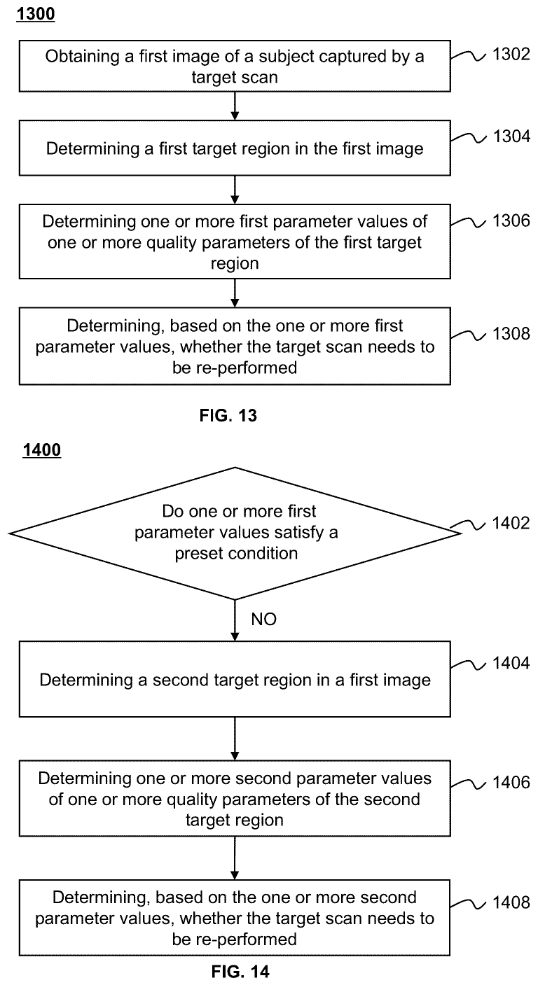

As another example, the processing device 140 may proceed to operation 312. In 312, the processing device 140 may further determine whether the target scan needs to be re-performed by analyzing the target image. For instance, the processing device 140 may determine a first target region in the target image. The processing device 140 may determine one or more first parameter values of one or more quality parameters of the first target region. Further, the processing device 140 may determine whether the target scan needs to be re-performed based on the one or more first parameter values. More descriptions regarding the determination of whether the target scan needs to be re-performed may be found in elsewhere in the present disclosure (e.g., FIGS. 13 and 14, and the descriptions thereof).

As still another example, after the processing device 140 determines that the target image includes image artifacts, the processing device 140 may proceed to operation 312 to determine whether the target scan needs to be re-performed based on a result of the artifact analysis. For example, the processing device 140 may determine a first area of artifact region(s) in the first target region and a second area of the first target region based on the result of the artifact analysis, and determine a proportion of artifact regions in the first target region based on the first area and the second area. The processing device 140 may further determine whether the target scan needs to be re-performed based on the proportion of artifact regions in the first target region.

According to some embodiments of the present disclosure, after the scout image is obtained, the one or more body parts of the subject corresponding to each bed position of the target scan may be determined, and the at least one scanning parameter or reconstruction parameter corresponding to each bed position may be determined based on the body part(s) corresponding to the bed position, which may reduce labor consumption and improve the efficiency of the target scan. In addition, by determining whether the target image includes image artifacts and/or whether the target scan needs to be re-performed, an image quality of the target scan may be improved.

It should be noted that the description of the process 300 is provided for the purposes of illustration, and is not intended to limit the scope of the present disclosure. For persons having ordinary skills in the art, various variations and modifications may be conducted under the teaching of the present disclosure. For example, operation 310 and/or operation 312 may be removed. However, those variations and modifications may not depart from the protection of the present disclosure.

FIG. 4 is a flowchart illustrating an exemplary process 400 for determining one or more body parts of a subject located at an i^{th} portion of a scanning table according to some embodiments of the present disclosure. The body part(s) located at the i^{th} portion of the scanning table may also be referred to as the body part(s) corresponding to the i^{th} bed position, and may be scanned in the i^{th} sub-scan of the target scan. In some embodiments, the process 400 may be performed to achieve at least part of operation 304 as described in connection with FIG. 3.

In 402, the processing device 140 (e.g., the determination module 220) may obtain a corresponding relationship between a plurality of feature points and a plurality of body part classifications.

A body part classification may refer to a type of a body part that one or more feature points belong to. Exemplary body part classifications may include the head, the chest, the abdomen, the pelvis, the lower extremity, etc. In some embodiments, each of the plurality of feature points may correspond to one body part classification. For example, the calvaria, the zygomatic bone, and the mandible may correspond to the head. The apex of the lung and the diaphragmatic dome may correspond to the chest. The femoral joint and the knee may correspond to the lower extremities.

In some embodiments, the corresponding relationship may be represented as a table, a diagram, a model, a mathematic function, or the like, or any combination thereof. In some embodiments, the corresponding relationship may be determined based on experience of a user (e.g., a technician, a doctor, a physicist, etc.). In some embodiments, the corresponding relationship may be determined based on a plurality of sets of historical data, wherein each set of the historical data may include a feature point and a corresponding body part classification. The historical data may be obtained by any measurement manner. For example, the corresponding relationship may be a classification model which is obtained by training an initial model based on the plurality of sets of historical data. As another example, the corresponding relationship may be determined based on classification rule(s) between the feature points and the body part classifications. In some embodiments, the processing device 140 may obtain the corresponding relationship from a storage device where the corresponding relationship is stored.

In 404, the processing device 140 (e.g., the determination module 220) may determine, based on a scout image, a positional relationship between the plurality of feature points and an i^{th} portion of the scanning table.

The positional relationship may indicate, for example, whether a feature point is located at the i^{th} portion of the scanning table, a shortest distance from the feature point to the i^{th} portion of the scanning table, etc. Since the subject is located the same position on the scanning table in the scout scan and the target scan, the positional relationship may be determined based on the scout image. In some embodiments, the processing device 140 may determine the positional relationship using an image recognition technique (e.g., an image recognition model, an image segmentation model, etc.) or based on information provided by a user (e.g., a doctor, an operator, a technician, etc.). For example, the processing device 140 may input the scout image of the subject to the image recognition model, and the image recognition model may output the positional relationship between the plurality of feature points and the i portion of the scanning table. The image recognition model may be obtained by training an initial model based on a plurality of training samples, wherein each of the plurality of training samples may include a sample scout image and the corresponding labelled sample scout image (i.e., the sample scout image is marked with a plurality of positioning boxes corresponding to the i^{th} portion of the scanning table). By using the image recognition model, the accuracy and efficiency of the determination of the positional relationship may be improved. As another example, the positional relationship may be firstly generated according to the image recognition technique, and then be adjusted or corrected by the user.

As still another example, the positional relationship may be determined by marking a plurality of positioning boxes corresponding to the N portions of the scanning table on the scout image. Merely by way of example, referring to FIG. 5C, FIG. 5C is a schematic diagram illustrating an exemplary scout image including feature points according to some embodiments of the present disclosure. As shown in FIG. 5C, positioning boxes 552, 554, 556, 558, and 560 are used to mark five image regions corresponding to five portions of the scanning table (or five bed positions of the target scan). Points 571, 572, 573, 574, 575, and 576 may be determined as feature points located at a first portion of the scanning table corresponding to the positioning box 552; points 576, 577, and 578 may be determined as feature points located at a second portion of the scanning table corresponding to the positioning box 554; points 579, 580, and 581 may be determined as feature points located at a third portion of the scanning table corresponding to the positioning box 556; points 582 and 583 may be determined as feature points located at a fourth portion of the scanning table corresponding to the positioning box 558, and points 584, 585, 586, 587, 588, and 589 may be determined as feature points located at a fifth portion of the scanning table corresponding to the positioning box 560.

In 406, the processing device 140 (e.g., the determination module 220) may determine, based on the corresponding relationship and the positional relationship, one or more body parts of the subject located at the i^{th} portion of the scanning table.

In some embodiments, the processing device 140 may determine, based on the positional relationship, one or more target feature points located at the i^{th} portion of the scanning table. Merely by way of example, as shown in FIG. 5C, points 571, 572, 573, 574, 575, and 576 may be determined as target feature points located at a first portion of the scanning table corresponding to the positioning box 552.

For each of the plurality of body part classifications, the processing device 140 may determine, based on the corresponding relationship, a count of target feature points that belong to the body part classification. For example, the processing device 140 may determine the body part classification of each target feature point based on the corresponding relationship, and then determine the count of target feature points that belong to each body part classification. For example, assuming that 4 target feature points are located at the i^{th} portion of the scanning table, the processing device 140 may determine 1 target feature point belongs to the body part classification of the head, and 3 target feature points belong to the body part classification of the chest.

Further, the processing device 140 may determine, based on the counts corresponding to the plurality of body part classifications, the one or more body parts of the subject located at the i^{th} portion of the scanning table. For example, if the count of the target feature points that belong to a specific body part classification is maximum, the processing device 140 may determine that the body part corresponding to the specific body part is located at the i^{th} portion of the scanning table. As another example, for each of the plurality of body part classifications, the processing device 140 may determine a ratio of the count of the target feature points that belong to the body part classification to a total count of the target feature points located at the i^{th} portion of the scanning table. Further, the processing device 140 may determine that the body part corresponding to the body part with a maximum ratio is located at the i^{th} portion of the scanning table.

Merely by way of example, referring to FIG. 5C, for the first portion of the scanning table corresponding to the positioning box 552, the points 571, 572, 573, 574, 575, and 576 located at the first portion belong to the body part classification of the head, so the count of the target feature points that belong to the body part classification of the head may be six. Correspondingly, it may be determined that the head of the subject is located at the first portion of the scanning table. Similarly, based on the body part classifications of the points 577-589, it may be determined that the chest of the subject is located at the second portion of the scanning table, the abdomen of the subject is located at the third portion of the scanning table, the pelvis of the subject is located at the fourth portion of the scanning table, and the lower extremities of the subject is located at the fifth portion of the scanning table.

In some embodiments, for each of the plurality of body part classifications, the processing device 140 may determine, based on the corresponding relationship, an axial distance between each two target feature points that belong to the body part classification. Further, the processing device 140 may determine, based on the distance between each two target feature points, the one or more body parts of the subject located at the i^{th} portion of the scanning table. For example, if the axial distance of two target feature points that belong to the body part classification satisfies a condition (e.g., larger than 60% of a length of the bed position), the body part classification may be determined as the body part of the subject located at the i^{th} portion of the scanning table.

In some embodiments, for each of the plurality of body part classifications, the processing device 140 may determine one or more key feature points that belong to the body part classification from the one or more target feature points based on the corresponding relationship. A key feature point may refer to a representative feature point representing a body part. For example, the calvaria and/or the zygomatic bone may be key feature points of the body part classification of the head. As another example, the apex of the lung and the diaphragmatic dome may be key feature points of the body part classification of the chest. Further, the processing device 140 may determine the one or more body parts of the subject located at the i^{th} portion of the scanning table based on the one or more key feature points corresponding to the one or more body part classifications. For example, referring to FIG. 5C, from the points 571-576 located at the first portion of the scanning table, the points 571 (i.e., the calvaria) and 572 (i.e., the zygomatic bone) are determined as the key feature points of the body part classification of the head. Accordingly, the processing device 140 may determine that the head of the subject is located at the first portion of the scanning table, and automatically select a head motion detection parameter as one scanning parameter corresponding to the first bed position. From the points 576-578 located at the second portion of the scanning table, the points 578 and 579 (i.e., the apex of the lung) are determined as the key feature points of the body part classification of the chest. Accordingly, the processing device 140 may determine that the chest of the subject is located at the second portion of the scanning table, and automatically select a respiratory motion detection parameter as one scanning parameter corresponding to the second bed position.

According to some embodiments of the present disclosure, for the i^{th} bed position, the one or more body parts of the subject located at the i^{th} portion of the scanning table may be determined automatically based on the plurality of feature points, which may improve the accuracy and efficiency of the determination of the one or more body parts, and in turn, improve the accuracy and efficiency of the target scan.

FIG. 7 is a flowchart illustrating an exemplary process 700 for determining whether a first image includes image artifacts according to some embodiments of the present disclosure. In some embodiments, the process 700 may be performed to achieve at least part of operation 310 as described in connection with FIG. 3. For example, the first image may be the target image as discussed in operation 310.

A medical image of a subject may include image artifacts due to multiple reasons. For example, a portion of the medical images may be abnormally bright due to metal implants in the subject and/or residues of drug injections. As another example, the medical image may include motion artifacts due to the respiratory motion, the heartbeat motion, the limb motion, etc. As still another example, the medical image may be truncated due to failures of a medical system. As a further example, a portion of the medical image may be blank due to the overestimation of a scatter correction coefficient. Due to the different reasons and different expressions of the image artifacts, it is difficult to reduce or eliminate the image artifacts automatically.

At present, the quality control of medical images normally relies on user intervention. For example, a user needs to inspect a medical image and determine whether the medical image includes image artifacts based on his/her own experience. In addition, a medical image including image artifacts may reduce the accuracy of diagnosis, and the medical image may need to be reprocessed and/or a medical scan may need to be re-performed to acquire a new medical image. In order to determine whether a medical image includes image artifacts and/or eliminate image artifacts in the medical image, the process 700 may be performed.

In 702, the processing device 140 (e.g., the obtaining module 210) may obtain the first image of a subject captured by a target scan and a second image of the subject captured by a reference scan. The target scan may be performed on the subject using a first imaging modality, and the reference scan may be performed on the subject using a second imaging modality. In some embodiments, the scanning parameters of the target scan may be set by performing operations 302-306. Alternatively, the scanning parameters of the target scan may be determined based on system default settings, set manually by a user, or determined according to the type of the subject.

The first image refers to an image of the subject that needs to be analyzed, for example, to determine whether the first image includes image artifacts. The second image refers an image of the subject other than the first image that provides reference information for facilitating the analysis of the first image. In some embodiments, the second imaging modality may be different from the first imaging modality. For example, the first imaging modality may be positron emission computed tomography (PET), and the second imaging modality may be computed tomography (CT) or magnetic resonance (MR). Correspondingly, the first image may be a PET image, and the second image may be a CT image or an MR image.

In some embodiments, the processing device 140 may obtain the first image from an imaging device for implementing the first imaging modality (e.g., a PET device, a PET scanner of a multi-modality imaging device, etc.) or a storage device (e.g., the storage device 150, a database, or an external storage) that stores the first image of the subject. Similarly, the processing device 140 may obtain the second image from an imaging device for implementing the second imaging modality (e.g., a CT device, an MRI scanner of a multi-modality imaging device, etc.) or a storage device (e.g., the storage device 150, a database, or an external storage) that stores the second image of the subject.

In 704, the processing device 140 (e.g., the generation module 230) may generate a third image based on the second image and an image prediction model. The third image may be a predicted image of the subject corresponding to the first imaging modality.

In some embodiments, the first image and the third image both correspond to the first imaging modality, but are generated in different manners. For instance, the third image may be generated by processing the second image based on the image prediction model, and the first image may be generated by performing the target scan on the subject using the first imaging modality. In other words, the first image may be a real image, and the third image may be a simulated image.

In some embodiments, the processing device 140 may input the second image to the image prediction model, and the image prediction model may output the third image corresponding to the first imaging modality.

In some embodiments, the image prediction model may include a first generation network. The first generation network may refer to a deep neural network that can generate an image corresponding to the first imaging modality based on an image corresponding to the second imaging modality. Exemplary first generation networks may include a generative adversarial network (GAN), a pixel recurrent neural network (PixelRNN), a draw network, a variational autoencoder (VAE), or the like, or any combination thereof. In some embodiments, the first generation network model may be part of a GAN model. The GAN model may further include a determination network (e.g., a neural network model).

In some embodiments, the image prediction model may be trained based on a plurality of first training samples and corresponding first labels. More descriptions regarding the generation of the image prediction model may be found in elsewhere in the present disclosure (e.g., FIG. 12 and the descriptions thereof).

In some embodiments, before the second image is input to the image prediction model, the processing device 140 may perform an artifact correction on the second image. For example, image artifacts in the second image may be corrected by performing thin-layer scanning, using a correction algorithm, etc. Since the third image is generated based on the second image, the image artifacts in the second image may reduce the accuracy of the third image. Therefore, performing the artifact correction on the second image may improve the accuracy of the third image.

In some embodiments of the present disclosure, by using the image prediction model, predicted functional images (e.g., PET images) with long acquisition time and large radiation doses may be generated based on anatomical images (e.g., CT images, MR images, etc.) that are easy to obtain and have low radiation doses. In other words, predicted functional images may be generated without bringing extra radiation exposure to the subject.

In 706, the processing device 140 (e.g., the determination module 220) may determine, based on the first image and the third image, whether the first image includes image artifacts.

In some embodiments, the processing device 140 may obtain a comparison result by comparing the first image and the third image, and then determine whether the first image includes the image artifacts based on the comparison result. For example, the comparison result may include a first similarity degree between the first image and the third image. Exemplary first similarity degrees may include a structural similarity (SSIM), a mean square error (MSE), or the like, or any combination thereof. Merely by way of example, the processing device 140 may determine the SSIM between the first image and the third image as the first similarity degree according to Equation (1):

$$SSIM ( x , y ) = \frac{( 2 {μ}_{x} \times {μ}_{y} + {c}_{1} ) ( 2 {σ}_{x y} + {c}_{2} )}{( {μ}_{x}^{2} + {μ}_{y}^{2} + {c}_{1} ) ( {σ}_{x}^{2} + {σ}_{y}^{2} + {c}_{2} )} , \qquad ( 1 )$$

where x represents the first image, y represents the third image, μₓ represents an average value of pixels in the first image, μ_{y} represents an average value of pixels in the third image, σₓ² represents a variance of the pixels in the first image, σ_{y}² represents a variance of the pixels in the third image, σ_{xy} represents a collaborative variance between the pixels in the first image and the pixels in the third image, c₁ and c₂ are constants for stabilizing the Equation (1), and SSIM(x,y) represents the first similarity degree between the first image and the third image. The value of the SSIM(x,y) may be within a range from −1 to 1. The larger the SSIM(x,y), the higher the first similarity degree between the first image and the third image may be.

As another example, the MSE between the first image and the third image may be determined as the first similarity degree according to Equation (2):

$$MSE ( x , y ) = \frac{1}{m n} {\sum }_{i = 1}^{n} {\sum }_{j = 1}^{m} {‖ x ( i , j ) - y ( i , j ) ‖}^{2} , \qquad ( 2 )$$

where m represents dimensions of the first image, n represents dimensions of the third image, and MSE(x,y) represents the first similarity degree between the first image and the third image. The less the MSE(x,y), the higher the first similarity degree between the first image and the third image may be.

In some embodiments, the processing device 140 may determine the first similarity degree based on a perceptual hash algorithm (PHA), a peak signal noise ratio algorithm, a histogram algorithm, etc. In some embodiments, the processing device 140 may determine the first similarity degree based on a trained machine learning model (e.g., a similarity degree determination model).

In some embodiments, the processing device 140 may determine whether the first similarity degree exceeds a first similarity threshold. The first similarity threshold may refer to a minimum value of the first similarity degree representing that the first image includes no image artifacts. The first similarity threshold may be determined based on system default setting or set manually by a user (e.g., a technician, a doctor, a physicist, etc.), such as, 0.6, 0.7, 0.8, 0.9, etc.

If the first similarity degree exceeds the first similarity threshold, the processing device 140 may determine that the first image includes no image artifacts. The processing device 140 may output the first image as a final image corresponding to the target scan. For example, the first image may be provided to the user for diagnosis. As another example, the first image may be stored in a storage device (e.g., the storage device 150), and may be retrieved based on a user instruction.

If the first similarity degree doesn't exceed the first similarity threshold, the processing device 140 may determine that the first image includes image artifacts. The processing device 140 may further proceed to operation 708.

In 708, the processing device 140 (e.g., the determination module 220) may determine one or more artifact regions of the first image. The one or more artifact regions may be represented by one or more target image blocks in the first image.

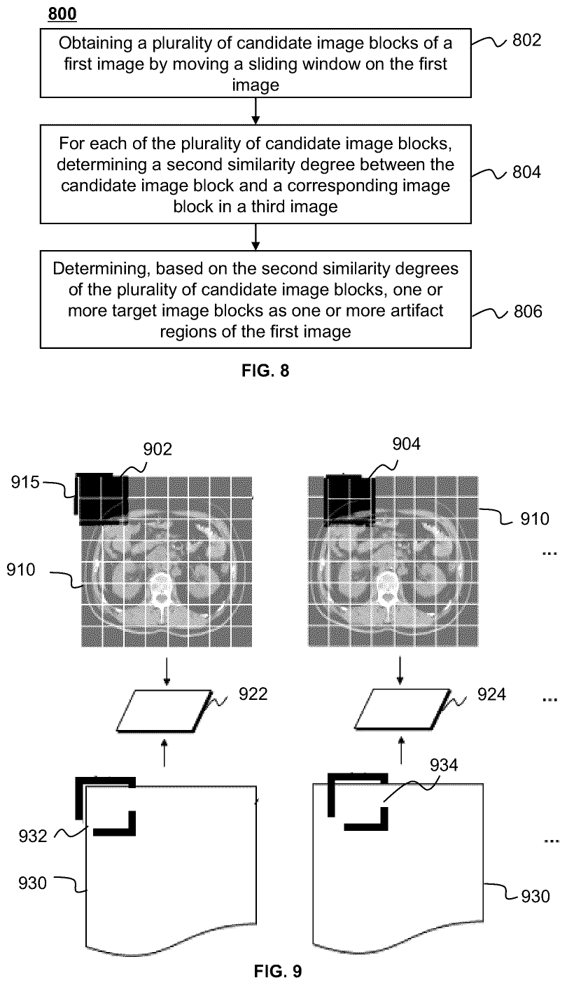

In some embodiments, the processing device 140 may determine the one or more artifact regions by segmenting the first image based on an image segmentation technique (e.g., an image segmentation model). In some embodiments, the processing device 140 may determine the one or more artifact regions of the first image using a sliding window technique. More descriptions regarding the determination of the one or more target image blocks using the sliding window technique may be found in elsewhere in the present disclosure (e.g., FIG. 8 and the descriptions thereof).

In 710, the processing device 140 (e.g., the generation module 230) may generate, based on the first image and the one or more target image blocks, one or more incomplete images.

An incomplete image may include a portion with no image data (i.e., a blank portion).

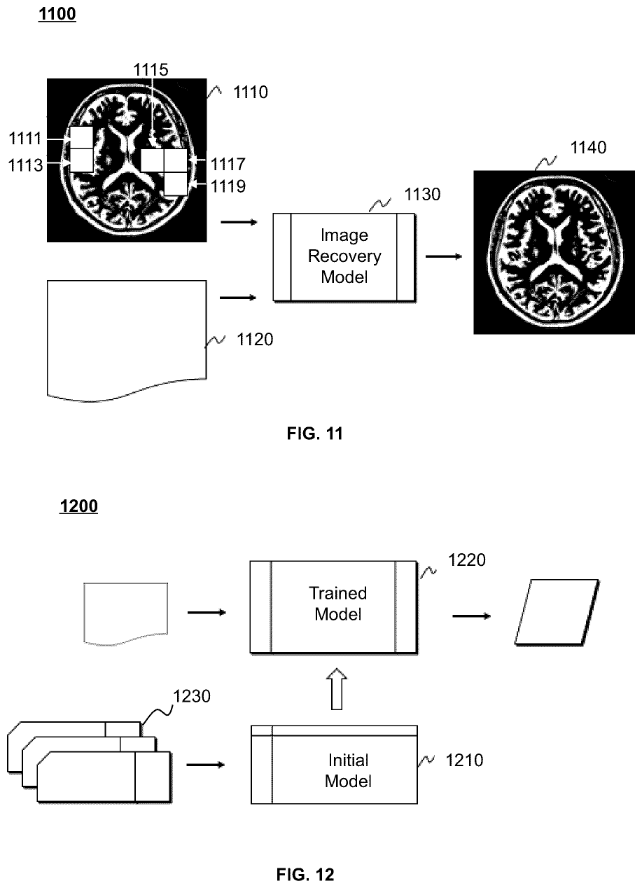

In some embodiments, the processing device 140 may generate an incomplete image by modifying at least a portion of the one or more target image blocks as one or more white image blocks. For example, the first image may include five target image blocks. The grey values of all pixels in the five target image blocks may be set to 255 to generate a single incomplete image. Merely by way of example, as shown in FIG. 11, an incomplete image 1110 includes five white image blocks 1111, 1113, 1115, 1117, and 1119 corresponding to the five target image blocks. As another example, the processing device 140 may modify the five target image blocks separately. Therefore, the processing device 140 may generate five incomplete images, and each of the five incomplete images may include one of the white image blocks 1111, 1113, 1115, 1117, and 1119.

In some embodiments, the processing device 140 may generate the one or more incomplete images in other manners, such as, modifying the one or more target image blocks as one or more black image blocks (i.e., designating gray values of pixels in the one or more target image blocks as 0), determining a boundary of a union of the one or more target image blocks, etc.

In 712, the processing device 140 (e.g., the generation module 230) may generate a corrected first image based on the one or more incomplete images and an image recovery model.

In some embodiments, the image recovery model may include may include a second generation network. The second generation network may refer to a deep neural network that can generate a corrected image by recovering one or more incomplete images. Exemplary second generation networks may include a generative adversarial network (GAN), a pixel recurrent neural network (PixelRNN), a draw network, a variational autoencoder (VAE), or the like, or any combination thereof. In some embodiments, the second generation network model may be part of a GAN model. The GAN model may further include a determination network (e.g., a neural network model). In some embodiments, the image recovery model may be trained based on a plurality of second training samples and corresponding labels. More descriptions regarding the model training may be found in elsewhere in the present disclosure (e.g., FIG. 12 and the descriptions thereof).

In some embodiments, the processing device 140 may input the one or more incomplete images into the image recovery model together, and the image recovery model may output the corrected first image. Incomplete regions (i.e., the one or more target image blocks) may be recovered through the image recovery model, and other regions (i.e., remaining candidate image blocks other than the one or more target image blocks in the first image) may be maintained. Therefore, the image correction may be performed on the artifact regions of the first image other than the whole first image, thereby reducing data volume of the image correction, and improving the efficiency of the image correction.

In some embodiments, each target image block in the first image may be modified separately to generate a corresponding incomplete image. If there are multiple target image blocks, a plurality of incomplete images may be generated. The processing device 140 may input the incomplete images into the image recovery model, respectively, to obtain multiple corrected images. The processing device 140 may further generate the corrected first image based on the multiple corrected images. By separately modifying each target image block in the first image, a portion of the corresponding incomplete image that needs to be recovered may be reduced, which may reduce the calculation amount of the image correction on each target image block and improve the efficiency of the image correction. In addition, since only information corresponding to one target image block is missing, the incomplete image corresponding to each target image block may include enough information for the image correction, which may improve the accuracy of the image correction.

In some embodiments, the image artifacts may appear on a sagittal slice, a coronal slice, and/or a transverse slice of the first image. For example, the identification and elimination of the image artifacts may be performed on the different slices of the first image, respectively. The one or more incomplete images may include normal image blocks (image blocks other than the target image blocks). The normal image blocks may be used to recover the blank image blocks of the incomplete image(s) so as to generate the corrected first image. Therefore, the lower the proportion of the blank image blocks in the incomplete image(s), the higher the accuracy of the corrected first image recovered by the image recovery model. If the image artifacts cover a large range in a certain direction (e.g., an image artifact exists in more than a certain count of continuous transverse slices), the sliding window may be placed on the first image along a sagittal direction and/or a coronal direction to determine the target image block(s) of the first image. In such cases, the input of the image recovery model may include incomplete image(s) corresponding to sagittal slice(s) and/or coronal slice(s). Correspondingly, the plurality of second training samples for training the image recovery model may correspond to a certain direction (e.g., the sagittal direction or the coronal direction). That is, if a sample incomplete image corresponding to a certain direction is used to train the image recovery model, an incomplete image corresponding to the same direction may be input into the image recovery model as an input. For example, if sample incomplete images corresponding to sample transverse slices are used to train the image recovery model, one or more incomplete images corresponding to one or more transverse slices of the first image may be input to the image recovery model as the input.

In some embodiments, the processing device 140 may input the one or more incomplete images (or a portion of the incomplete image(s)) and the second image into the image recovery model. The image correction may be performed based on the one or more incomplete images and the second image (e.g., the anatomical image, such as a CT image, an MR image, etc.). Since the second image can provide additional reference information, the accuracy and the efficiency of the image recovery may be improved.

Merely by way of example, referring to FIG. 11 again, FIG. 11 is a schematic diagram illustrating an exemplary process 1100 for generating a corrected first image according to some embodiments of the present disclosure.

As shown in FIG. 11, the incomplete image 1110 may include blank image blocks 1111, 1113, 1115, 1117, and 1119. The incomplete image 1110 and a second image 1120 may be input to an image recovery model 1130, and the image recovery model 1130 may output a corrected first image 1140. In the corrected first image 1140, the blank image blocks 1111, 1113, 1115, 1117, and 1119 may be recovered. In some embodiments, the second image 1120 may be omitted.

In some embodiments, the corrected first image may be further processed. For example, the processing device 140 may smooth edges of corrected region(s) corresponding to the artifact region(s).

According to some embodiments of the present disclosure, the third image corresponding to the first imaging modality may be generated based on the second image corresponding to the second imaging modality and the image prediction model, and then whether the first image includes image artifacts may be determined automatically based on the first image and the third image. Compared to a conventional way that a user needs to manually determine whether the first image includes the image artifacts, the automated imaging systems and methods disclosed herein may be more accurate and efficient by, e.g., reducing the workload of the user, cross-user variations, and the time needed for image artifact analysis.

Further, if the first image includes the image artifacts, the image artifacts may be automatically corrected using the image recovery model, which may improve the efficiency of the image correction.

FIG. 8 is a flowchart illustrating an exemplary process 800 for determining one or more target image blocks according to some embodiments of the present disclosure. In some embodiments, the process 800 may be performed to achieve at least part of operation 708 as described in connection with FIG. 7.

In 802, the processing device 140 (e.g., the determination module 220) may obtain a plurality of candidate image blocks of a first image by moving a sliding window on the first image.

A candidate image block (also referred to as a sub-image block) may be a portion of the first image that has the same size and shape as the sliding window. In some embodiments, the first image may include the plurality of candidate image blocks. The sizes of the plurality of candidate image blocks may be the same, and the positions of the plurality of candidate image blocks in the first image may be different. In some embodiments, if there are no other candidate image blocks between two candidate image blocks in a certain direction, the two candidate image blocks may be adjacent in the certain direction. The two candidate image blocks may be referred to as adjacent candidate image blocks. In some embodiments, adjacent candidate image blocks of the plurality of candidate image blocks may be overlapping or not overlapping. In some embodiments, two adjacent candidate image blocks may contact with each other. In some embodiments, the union of the plurality of candidate image blocks may form the first image.

In some embodiments, the processing device 140 may move the sliding window on the first image to obtain the candidate image blocks. For example, if the first image includes 256×256 pixels, a size of the sliding window is 64×64 pixels, and a sliding distance of the sliding window in a horizontal or vertical direction is 32 pixels, 49 (i.e., 7×7) candidate image blocks may be obtained.

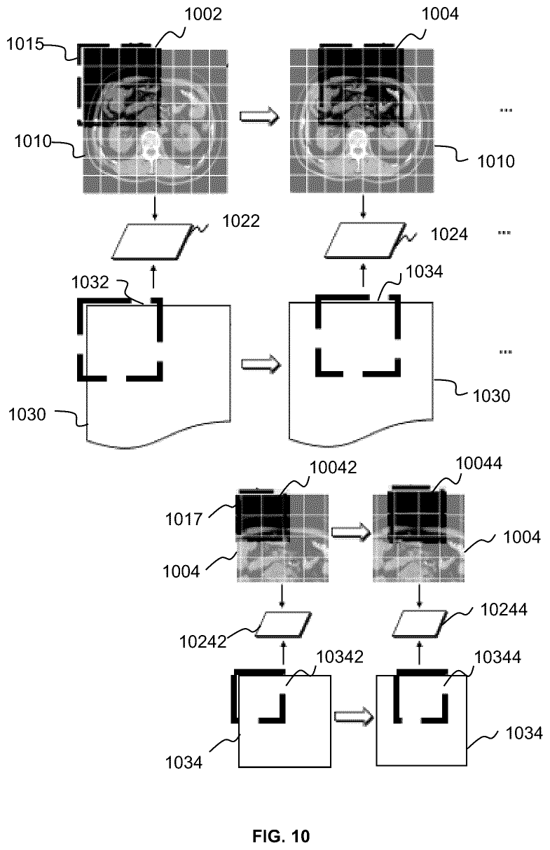

In some embodiments, the processing device 140 may move a plurality of sliding windows having different sizes (or referred to as multilevel sliding windows) on the first image to obtain the candidate image blocks of the first image. More descriptions regarding the determination of the candidate image blocks may be found in elsewhere in the present disclosure (e.g., FIG. 10 and the descriptions thereof).

In 804, for each of the plurality of candidate image blocks, the processing device 140 (e.g., the determination module 220) may determine a second similarity degree between the candidate image block and a corresponding image block in a third image.

The corresponding image block in the third image may refer to an image block whose relative position in the third image is the same as a relative position of the candidate image block in the first image. For example, each pixel of the first image and each pixel of the third image may be represented by coordinates. If the candidate image block includes a first pixel having a specific coordinate, the corresponding image block may include a second pixel also having the specific coordinate.

In some embodiments, the determination of the second similarity degree between the candidate image block and the corresponding image block may be similar to the determination of the first similarity degree between the first image and the third image. For example, the second similarity degree may be determined according to Equation (1) and/or Equation (2). As another example, the processing device 140 may determine the second similarity degree using a trained machine learning model (e.g., a similarity degree determination model). In some embodiments, the similarity degree determination model may be a portion of the image recovery model. In some embodiments, the similarity degree determination model and the image recovery model may be two separate models.

In 806, the processing device 140 (e.g., the determination module 220) may determine, based on the second similarity degrees of the candidate image blocks, one or more target image blocks as one or more artifact regions of the first image.

A target image block may refer to an image block including image artifacts. In some embodiments, for each of the plurality of candidate image blocks, the processing device 140 may determine whether the second similarity degree between the candidate image block and the corresponding image block satisfies a condition. The condition may refer to a pre-set condition for determining whether a candidate image block is a target image block. For example, the condition may be that the second similarity degree is below a second similarity threshold. The second similarity threshold may be determined based on system default setting (e.g., statistic information) or set manually by a user (e.g., a technician, a doctor, a physicist, etc.), such as, 0.6, 0.7, 0.8, 0.9, etc. For example, for one of the plurality of candidate image blocks, if the second similarity degree between the candidate image block and the corresponding image block doesn't satisfy the condition (e.g., doesn't exceed 0.8), the candidate image block may be determined as a target image block. If the second similarity degree between the candidate image block and the corresponding image block satisfies the condition (e.g., exceeds 0.8), the candidate image block may not be determined as a target image block.

In some embodiments, the processing device 140 may further process the one or more target image blocks. For example, the processing device 140 may correct the one or more target image blocks.

For illustration purposes, FIGS. 9 and 10 are provided to illustrate exemplary processes for determining one or more target image blocks according to some embodiments of the present disclosure.

As shown in FIG. 9, the processing device 140 may obtain one or more target image blocks of a first image 910 by moving a sliding window (denoted by a black box 915 in FIG. 9) on the first image 910. If the first image 910 includes 256×256 pixels, a size of the sliding window is 64×64 pixels, and a sliding distance of the sliding window in a horizontal or vertical direction (e.g., sliding from a candidate image block 902 to a candidate image block 904) is 32 pixels, 49 (i.e., 7×7) candidate image blocks may be obtained.

For each of the 49 candidate image blocks, a second similarity degree between the candidate image block and a corresponding image block in a third image 930 may be determined. For example, a second similarity degree 922 between the candidate image block 902 and the corresponding image block 932 may be determined. As another example, a second similarity degree 924 between the candidate image block 904 and the corresponding image block 934 may be determined. For each of the 49 candidate image blocks, if the second similarity degree between the candidate image block and the corresponding image block in the third image 930 doesn't satisfy the condition (e.g., doesn't exceed 0.8), the candidate image block may be determined as a target image block to be further processed. If the second similarity degree between the candidate image block and the corresponding image block in the third image 930 satisfies the condition (e.g., exceeds 0.8), the candidate image block may not be determined as the target image block and omitted from further processing. For example, if the second similarity degree 922 is 0.9 that exceeds 0.8, the candidate image block 902 may not be determined as a target image block. As another example, if the second similarity degree 924 is 0.7 that doesn't exceed 0.8, the candidate image block 904 may be determined as a target image block to be further processed (e.g., corrected).

As another example, referring to FIG. 10, the processing device 140 may obtain one or more target image blocks of a first image 1010 by moving a first-level sliding window (denoted by a black box 1015 in FIG. 10) and a second-level sliding window (denoted by a black box 1017 in FIG. 10) on the first image 1010.

If the first image 1010 includes 256×256 pixels, a size of a first-level sliding window is 128×128 pixels, and a sliding distance of the first-level sliding window in a horizontal or vertical direction (e.g., sliding from a first-level candidate image block 1002 to a first-level candidate image block 1004) is 32 pixels, 9 (i.e., 3×3) first-level candidate image blocks may be obtained.

For each of the 9 first-level candidate image blocks, a second similarity degree between the first-level candidate image block and a corresponding first-level image block in a third image 1030 may be determined. For example, a second similarity degree 1022 between the first-level candidate image block 1002 and the corresponding first-level image block 1032 may be determined. As another example, a second similarity degree 1024 between the first-level candidate image block 1004 and the corresponding first-level image block 1034 may be determined. For each of the 9 first-level candidate image blocks, if the second similarity degree between the first-level candidate image block in the first image 1010 and the corresponding first-level image block in the third image 1030 doesn't satisfy the condition (e.g., doesn't exceed the second similarity threshold (e.g., 0.8)), the first-level candidate image block may be determined as a preliminary target image block including the image artifacts. If the second similarity degree between the first-level candidate image block in the first image 1010 and the corresponding first-level image block in the third image 1030 satisfies the condition, the first-level candidate image block may not be determined as the preliminary target image block and omitted from further processing. For example, if the second similarity degree 1022 is 0.9 that exceeds 0.8, the first-level candidate image block 1002 may not be determined as the preliminary target image block. As another example, if the second similarity degree 1024 is 0.7 that doesn't exceed 0.8, the first-level candidate image block 1004 may be determined as the preliminary target image block including the image artifacts.

Further, the preliminary target image block including the image artifacts may be determined as a plurality of second-level candidate image blocks. For example, if a second-level sliding window includes 64×64 pixels, and a sliding distance of the second-level sliding window in a horizontal or vertical direction (e.g., sliding from a second-level candidate image block 10042 to a second-level candidate image block 10044) is 32 pixels, 9 (i.e., 3×3) second-level candidate image blocks may be obtained.

For each of the 9 second-level candidate image blocks, a second similarity degree between the second-level candidate image block and a corresponding second-level image block in the third image 1030 may be determined. The determination of the second similarity degree between the second-level candidate image block and the corresponding second-level image block in the third image 1030 may be similar to the determination of the second similarity degree between the first-level candidate image block and the corresponding first-level image block in the third image 1030. Further, for each of the 9 second-level candidate image blocks, if the second similarity degree between the second-level candidate image block in the first image 1010 and the corresponding second-level image block in the third image 1030 doesn't satisfy the condition (e.g., doesn't exceed the second similarity threshold), the second-level candidate image block may be determined as a target image block including the image artifacts to be further processed (e.g., corrected). If the second similarity degree between the second-level candidate image block in the first image 1010 and the corresponding second-level image block in the third image 1030 satisfies the condition, the second-level image block may not be determined as the target image block and omitted from further processing. For example, a second similarity degree 10242 between the second-level candidate image block 10042 and the corresponding second-level image block 10342 may be determined. As another example, a second similarity degree 10244 between the second-level candidate image block 10044 and the corresponding second-level image block 10344 may be determined. Further, if the second similarity degree 10242 is 0.9 that exceeds 0.8, the second-level candidate image block 10042 may not be determined as the target image block. If the second similarity degree 10244 is 0.7 that doesn't exceed 0.8, the second-level candidate image block 10044 may be determined as the target image block including image artifacts. In some embodiments, the second similarity threshold may be the same as or different from the first similarity threshold.

According to some embodiments of the present disclosure, the sliding window may be used to position the image artifacts with a fine granularity. In addition, a multi-level sliding window (e.g., the first-level sliding window and the second-level sliding window) may be used to screen a large region in the first image and then position the image artifacts in the large region, which may reduce the workload.

FIG. 12 is a schematic diagram illustrating an exemplary process 1200 for training an initial model according to some embodiments of the present disclosure.

As shown in FIG. 12, a trained model 1220 may be obtained by training an initial model 1210 based on a plurality of training samples 1230. In some embodiments, the initial model 1210 may include an initial image prediction model and/or an initial image recovery model, and the trained model 1220 may include an image prediction model and/or an image recovery model.

In some embodiments, the image prediction model may be obtained by training the initial image prediction model based on a plurality of first training samples. A first training sample may include image data for training the initial image prediction model. For example, the first training sample may include historical image data.

In some embodiments, each of the plurality of first training samples may include a sample second image of a sample subject as an input of the initial image prediction model, and a sample first image of the sample subject as a first label. The sample first image may be obtained by scanning the sample subject using the first imaging modality, and the sample second image may be obtained by scanning the sample subject using the second imaging modality. In some embodiments, the first imaging modality may be PET, and the second imaging modality may be CT or MR. Correspondingly, the sample first image may be a sample PET image, and the sample second image may be a sample CT image or a sample MR image.

In some embodiments, the processing device 140 may obtain the plurality of first training samples by retrieving (e.g., through a data interface) a database or a storage device.

During the training of the initial image prediction model, the plurality of first training samples may be input to the initial image prediction model, and first parameter(s) of the initial image prediction model may be updated through one or more iterations. For example, the processing device 140 may input the sample second image of each first training sample into the initial image prediction model, and obtain a prediction result. The processing device 140 may determine a loss function based on the prediction result and the first label (i.e., the corresponding sample first image) of each first training sample. The loss function may refer to a difference between the prediction result and the first label. The processing device 140 may adjust the parameter(s) of the initial image prediction model based on the loss function to reduce the difference between the prediction result and the first label. For example, by continuously adjusting the parameter(s) of the initial image prediction model, the loss function value may be reduced or minimized.

In some embodiments, the image prediction model may also be obtained according to other training manners. For example, an initial learning rate (e.g., 0.1), an attenuation strategy, etc., corresponding to the initial image prediction model may be determined, and the image prediction model may be obtained based on the initial learning rate (e.g., 0.1) and the attenuation strategy, etc., using the plurality of first training samples.

In some embodiments, the image recovery model may be obtained by training the initial image recovery model based on a plurality of second training samples.

A second training sample may include image data for training the initial image recovery model. For example, the second training sample may include historical image data.

In some embodiments, each of the plurality of second training samples may include a sample incomplete image of a sample subject as an input of the initial image recovery model, and a sample image of the sample subject as a second label. The sample image may be obtained by scanning the sample subject using the first imaging modality.

In some embodiments, the sample incomplete image may be generated by removing a portion of image data from the sample image. For example, the sample incomplete image may be obtained by adding a mask on the sample image. After the mask is added, gray values in mask region(s) of the sample image may be set to 0 (or 255). That is, the mask region(s) of the sample image may be covered with one or more completely black (or completely white) opaque image blocks in the visual effect. In some embodiments, a shape and size of the mask may be related to a candidate image block. For example, the shape and size of the mask may be the same as that of the candidate image block. As another example, a horizontal length of the mask may be 1.5 times, 2 times, etc., a horizontal length of the candidate image block. As still another example, a vertical length of the mask may be 1.5 times, 2 times, etc., a vertical length of the candidate image block. The mask may be a combination of one or more candidate image blocks. For example, the mask may include at least two adjacent candidate image blocks or at least two independent candidate image blocks. More descriptions regarding the candidate image block may be found in elsewhere in the present disclosure (e.g., FIGS. 8-10 and the descriptions thereof).

In some embodiments, a position of the mask may be set randomly in the sample image. In some embodiments, the position of the mask may be set based on default rule(s).

In some embodiments, multiple second training samples may be obtained by setting up different masks on the sample image. For example, a second training sample 1 may be a sample incomplete image 1 including a sample image A and a mask 1, and a second training sample 2 may be a sample incomplete image 2 including the sample image A and a mask 2. Labels of the second training sample 1 and the second training sample 2 may be the sample image A.

In some embodiments, the first imaging modality may be PET. Correspondingly, the sample incomplete image may be an incomplete sample PET image, and the sample image may be a sample PET image.

In some embodiments, the processing device 140 may obtain the plurality of second training samples by retrieving (e.g., through a data interface) a database or a storage device.

During the training of the initial image recovery model, the plurality of second training samples may be input to the initial image recovery model, and second parameter(s) of the initial image recovery model may be updated through one or more iterations. For example, the processing device 140 may input the sample incomplete image of each second training sample into the initial image recovery model, and obtain a recovery result.

The processing device 140 may determine a loss function based on the recovery result and the second label (i.e., the corresponding sample image) of each second training sample. The loss function may refer to a difference between the recovery result and the second label. The processing device 140 may adjust the parameter(s) of the initial image recovery model based on the loss function to reduce the difference between the recovery result and the second label. For example, by continuously adjusting the parameter(s) of the initial image recovery model, the loss function value may be reduced or minimized.

In some embodiments, the image recovery model may also be obtained according to other training manners.

In some embodiments, the initial model 1210 may include an initial generator and an initial discriminator. The initial generator and the initial discriminator may be jointly trained based on the plurality of training samples 1230. In some embodiments, the trained generator may be determined as the trained model 1220 (e.g., the image prediction model and/or the image recovery model).

In some embodiments, the generation of the trained model 1220 described in FIG. 12 and the use of the trained model 1220 (e.g., the generation of the incomplete image and the corrected first image described in FIGS. 7-8) may be executed on different processing devices. For example, the generation of the trained model 1220 described in FIG. 12 may be performed on a processing device of a manufacturer of the imaging device, and the use of a portion or all of the trained model 1220 may be performed on a processing device (e.g., the processing device 140) of a user (e.g., hospitals) of the imaging device.

FIG. 13 is a flowchart illustrating an exemplary process 1300 for determining whether a target scan needs to be re-performed according to some embodiments of the present disclosure. In some embodiments, the process 1300 may be performed to achieve at least part of operation 312 as described in connection with FIG. 3.

When a medical imaging device, such as a nuclear medical device (e.g., a PET device, a SPECT device, etc.) scans a subject, the quality of the scanned images may be uneven, and a user (e.g., doctors, operators, technicians, etc.) needs to determine whether the scan needs to be re-performed based on experience. However, the efficiency and accuracy of the manual determination are low. In order to improve the efficiency and accuracy of the determination of whether the scan needs to be re-performed, the process 1300 may be performed.

In 1302, the processing device 140 (e.g., the obtaining module 210) may obtain a first image of a subject captured by a target scan.

In some embodiments, the obtaining of the first image may be similar to the obtaining of the first image described in operation 702.

In some embodiments, the processing device 140 may obtain the first image from an imaging device for implementing a first imaging modality (e.g., a PET device, a PET scanner of a multi-modality imaging device, etc.) or a storage device (e.g., the storage device 150, a database, or an external storage) that stores the first image of the subject.

In 1304, the processing device 140 (e.g., the determination module 220) may determine a first target region in the first image.

The first target region may refer to a region used to evaluate the quality of the first image. In some embodiments, the first target region may include region(s) of one or more typical organs and/or tissues of the subject where the uptake of radionuclides is uniform. In some embodiments, the first target region may include a liver region, an aortic blood pool, an ascending aorta/descending aorta, a gluteal muscle region, a brain region, or the like, or any combination thereof.

In some embodiments, the first target region may be obtained by identifying the first target region from the first image through an image recognition model. In some embodiments, the first target region may be obtained by segmenting the first image using an image segmentation model.

In some embodiments, the first target region may be obtained based on a corresponding region of the typical organ(s) and/or tissue(s) of the subject in a second image.

In some embodiments, the processing device 140 may obtain the second image (e.g., a CT image, an MR image, etc.) of the subject. For example, the processing device 140 may obtain the second image from an imaging device for implementing a second imaging modality (e.g., a CT device, an MR scanner of a multi-modality imaging device, etc.) or a storage device (e.g., the storage device 150, a database, or an external storage) that stores the second image of the subject.

The processing device 140 may further identify a first region from the second image. The first region may refer to a region representing one or more typical organs and/or tissues of the subject and correspond to the first target region in the second image. For example, the first target region may be the liver region in the first image, and the first region may be the liver region in the second image.

In some embodiments, the processing device 140 may identify the first region in the second image through a machine learning model. An input of the machine learning model may be the second image, and an output of the machine learning model may be the second image in that the first region is marked or a segmentation mask indicating the first region. In some embodiments, the machine learning model may be obtained by training a neural network model e.g., a graph neural network (GNN)). For example, the machine learning model may be a trained neural network model, and stored in the imaging system 100 (e.g., the processing device 140, the storage device 150, etc.) through an interface. In some embodiments, the machine learning model may be a deep learning model. For example, the machine learning models may be obtained by training a 3D V-net.

In some embodiments, before training the machine learning model, training sample data may be preprocessed. For example, a sample image of the liver region may be enhanced (e.g., through a contrast limited adaptive histogram equalization (CLAHE) technique), and a size of the sample image may be adjusted to 256×256. As another example, the sample image of the liver region and corresponding label data of the liver region may be stored in a preset format (e.g., .nii format) using a processing tool (e.g., an itk tool).

In some embodiments, the machine learning model may be obtained by training a 2.5D V-net. Requirements on hardware (e.g., a graphics processing unit (GPU)) for training the 2.5D V-net may be reduced compared to requirements on hardware for training the 3D V-net. In addition, channel information of the first image may be fully used through the 2.5D V-net. For example, a size of input data of the 2.5D V-net may be [256,256,64]. The input data may be processed by a first branch and a second branch of the 2.5D V-net. The first branch may be used to perform a convolution operation in a channel direction. A size of a convolution core of the first branch may be 1×1. The second branch may be used to perform a convolution operation in an XY surface. A size of a convolution core of the second branch may be 3×3. The outputs of the two branches may be merged in the channel direction for a next sampling operation.

In some embodiments, the processing device 140 may determine the first target region in the first image based on the first region segmented from the second image. In some embodiments, the processing device 140 may determine the first target region in the first image by mapping the first region to the first image through a registration matrix. The registration matrix may refer to a transfer matrix that converts a second coordinate system corresponding to the second image to a first coordinate system corresponding to the first image. The registration matrix may be used to transform coordinate information of the first region into coordinate information of the first target region.

Since structural information of the subject included in the second image (e.g., a structural image such as a CT image) is richer than structural information of the subject included in the first image (e.g., a functional image such as the PET image), using the machine learning model to identify the first region in the second image and subsequent identifying the first target region in the first image based on the first region may improve the efficiency and accuracy of identifying the first target region.

In 1306, the processing device 140 (e.g., the determination module 220) may determine one or more first parameter values of one or more quality parameters of the first target region.

A quality parameter may be used to measure the image quality. In some embodiments, the one or more quality parameters may include a signal noise ratio (SNR), a proportion of artifact regions in a target region (e.g., the first target region, the second target region, etc.), a resolution, a contrast, a sharpness, etc.

The SNR may be used to compare the level of desired signals to the level of noises in the first target region. In some embodiments, the SNR may refer to a ratio of signals power to the noise power in the first target region. In some embodiments, the processing device 140 may determine a first parameter value of the SNR of the first target region through an approximately estimation. For example, the processing device 140 may determine a ratio of a variance of the signals in the first target region to a variance of the noises in the first target region. Merely by way of example, the processing device 140 may determine a local variance of each pixel in the first target region. The processing device 140 may designate a maximum value among the local variances as the variance of the signals in the first target region, and designate a minimum value among the local variances as the variance of the noises in the first target region. The ratio of the variance of the signals to the variance of the noises in the first target region may be determined. Further, the first parameter value of the SNR of the first target region may be determined by adjusting the ratio based on an experience formula. In some embodiments, the processing device 140 may also determine the first parameter value of the SNR of the first target region in other manners, which is not limited herein.

The proportion of the artifact regions in the first target region may refer to a ratio of an area of the artifact regions in the first target region to an area of the whole first target region. In some embodiments, the processing device 140 may determine the area of the artifact regions in the first target region based on a result of the artifact analysis by performing operations 702-708, and then determine the proportion of the artifact regions in the first target region based on the area of the artifact regions and the area of the first target region. In some embodiments, the processing device 140 may determine the proportion of the artifact regions in the first target region in other manners, which is not limited herein.

In some embodiments, the one or more first parameter values of the one or more quality parameters (e.g., the SNR, the proportion, etc.) of the first target region and the first image (e.g., the PET image) may be loaded and displayed on a display interface, simultaneously, for a user to read.

In 1308, the processing device 140 (e.g., the determination module 220) may determine, based on the one or more first parameter values, whether the target scan needs to be re-performed.

The re-performing the target scan may include performing a supplementary scan and/or performing a re-scan on the subject.

The supplementary scan may be performed on the subject after the target scan to extend the total scan time of the subject. Correspondingly, supplementary scanning data corresponding to the supplementary scan may be obtained. For example, the target scan may last for 3 minutes, and current scanning data may be obtained by the target scan. The first image may be generated based on the current scanning data. Then, operations 1302-1308 may be performed on the first image, and the processing device 140 may determine that a supplementary scan needs to be performed. After the supplementary scan is performed on the subject for 2 minutes, supplementary scanning data may be obtained, and a new first image may be re-generated based on the current scanning data and the supplementary scanning data.

The re-scan may refer to that the target scan is re-performed on the subject. Correspondingly, re-scanning data may be obtained. For example, after operations 1302-1308 are performed on the first image, the processing device 140 may determine that the target scan needs to be re-performed. After the re-scan is performed on the subject, re-scanning data may be obtained, and the first image may be re-generated based on the re-scanning data. In some embodiments, scanning parameter(s) (e.g., a scanning time) of the re-scan may be the same as or different from scanning parameter(s) of the target scan.

In some embodiments, the processing device 140 may determine whether the target scan needs to be re-performed based on the one or more first parameter values. For example, the processing device 140 may determine whether the one or more first parameter values satisfy a first preset condition. If the one or more first parameter values don't satisfy the first preset condition, the processing device 140 may determine that the target scan needs to be re-performed. If the one or more first parameter values satisfy the first preset condition, the processing device 140 may determine that the target scan doesn't need to be re-performed.

Merely by way of example, the processing device 140 may determine that the target scan needs to be re-performed when the first parameter value of the SNR of the first target region doesn't satisfy a first SNR threshold. In some embodiments, the processing device 140 may determine a time for a supplementary scan based on a difference between the first parameter value of the SNR of the first target region and the first SNR threshold. For example, if the difference between the first parameter value of the SNR of the first target region and the first SNR threshold is 2, the time for the supplementary scan may be 1 minute. If the difference between the first parameter value of the SNR of the first target region and the first SNR threshold is 4, the time for the supplementary scan may be 3 minutes. In some embodiments, a plurality of reference ranges may be set for the difference between the value of the SNR of the first target region and the first SNR threshold. The processing device 140 may determine a time for a supplementary scan based on the difference and the reference ranges. For example, if the difference is within a range from 2 to 4, the time for the supplementary scan may be 1 minute. If the difference is within a range from 4 to 6, the time for the supplementary scan may be 3 minutes. If the difference exceeds a re-scan threshold (e.g., 10), the processing device 140 may determine that a re-scan may need to be performed on the subject.

In some embodiments, the supplementary scan or the re-scan may be determined based on a per-set scan parameter. For example, if the requirements on the image quality are high, the processing device 140 may determine that the re-scan needs to be performed on the subject when the one or more first parameter values don't satisfy the preset condition. As another example, considering the image quality and the scanning efficiency, the processing device 140 may determine that the supplementary scan needs to be performed on the subject when the one or more first parameter values don't satisfy the preset condition.

According to some embodiments of the present disclosure, whether the target scan needs to be re-performed may be determined based on a determination of whether one or more first parameter values satisfy the first preset condition, which may involve simple calculation and improve the accuracy of the determination of whether the target scan needs to be re-performed.

In some embodiments, if the one or more first parameter values don't satisfy the preset condition, the processing device 140 may determine a second target region in the first image, and determine one or more second parameter values of the one or more quality parameters of the second target region. Further, the processing device 140 may determine, based on the one or more second parameter values, whether the target scan needs to be re-performed. More descriptions regarding the determination of whether the target scan needs to be re-performed may be found in elsewhere in the present disclosure (e.g., FIG. 14 and the descriptions thereof).

In some embodiments, the target scan may be re-performed on the first target region. In some embodiments, the target scan may be re-performed on at least one bed position corresponding to the first target region. In some embodiments, the target scan may be re-performed on the whole subject for re-generating the first image.

In some embodiments, if the target scan needs to be re-performed, the processing device 140 may send a prompt.

The prompt may refer to information that can prompt a user who uses the imaging system 100 provided by some embodiments of the present disclosure. The prompt may be in the form of an image, text, a sound, a vibration, or the like, or any combination thereof. In some embodiments, the prompt may be sent to the user through the terminal 130. For example, the prompt (e.g., the image, the text, etc.) may be sent through a display screen of the terminal 130. As another example, a vibration prompt may be sent through a vibration component of the terminal 130. As still another example, a sound prompt may be sent through a speaker of the terminal 130. In some embodiments, the prompt may include information such as, a time for the supplementary scan, scanning parameter(s) of the re-scan, a portion of the subject that needs to receive the re-scan/the supplementary scan, reasons for the re-scan/the supplementary scan, or the like, or any combination thereof.

In some embodiments, if the one or more first parameter values satisfy the preset condition, the processing device 140 may continue to scan a next bed position based on the scanning parameter(s) or end the target scan.

In some embodiments, the processing device 140 may perform operations 1302-1308 after completing the target scan (e.g., a PET scan) of the whole subject. In some embodiments, the processing device 140 may perform operations 1302-1308 before the target scan is completed, for example, after a portion of the subject (e.g., a scanning region corresponding to a specific bed position, the upper body, the lower body, etc.) is scanned.

According to some embodiments of the present disclosure, whether the target scan needs to be re-preformed may be automatically determined based on the one or more first parameter values of the one or more quality parameters of the first target region, which may reduce the labor consumption and the dependence on the experience of the user, and improve the efficiency of the determination of whether the target scan needs to be re-preformed. Since the first target region includes the region(s) of the one or more typical organs and/or tissues of the subject where the uptake of radionuclides is uniform, the one or more quality parameters of the first target region may have a relevantly higher reference value, which may improve the accuracy of the determination of whether the target scan needs to be re-preformed. In addition, whether the target scan needs to be re-preformed may be determined and the prompt may be sent during the target scan without performing an additional scan after the target scan, which may save the operation time, shorten the scanning time, and improve the user experience.

FIG. 14 is a flowchart illustrating an exemplary process 1400 for determining whether a target scan needs to be re-performed according to some embodiments of the present disclosure. In some embodiments, the process 1400 may be performed to achieve at least part of operation 1308 as described in connection with FIG. 13.

In 1402, the processing device 140 (e.g., the determination module 220) may determine whether the one or more first parameter values satisfy a first preset condition.

The first preset condition may include that a first parameter value of the SNR of the first target region exceeds or reaches a first SNR threshold, a first parameter value of a proportion of artifact regions in the first target region doesn't exceed a first proportion threshold, etc. In some embodiments, the first SNR threshold and/or the first proportion threshold may be determined based on system default setting (e.g., statistic information) or set manually by a user (e.g., a technician, a doctor, a physicist, etc.). For example, the first SNR threshold and/or the first proportion threshold may be input by a user through the terminal 130 and stored in the storage device 150.

If the one or more first parameter values satisfy the first preset condition, the processing device 140 may determine the first image as an acceptable image, and a target scan may not need to be re-performed. For example, if the first parameter value of the SNR of the first target region exceeds or reaches the first SNR threshold, and the first parameter value of the proportion of the artifact regions in the first target region doesn't exceed the first proportion threshold, the processing device 140 may determine the first image as an acceptable image. As another example, if the first parameter value of the SNR of the first target region exceeds or reaches the first SNR threshold, or the first parameter value of the proportion of the artifact regions in the first target region doesn't exceed the first proportion threshold, the processing device 140 may determine the first image as the acceptable image.

In some embodiments, the one or more quality parameters may include an SNR of the first target region. The SNR of the first target region may be affected by various factors, such as noises, lesions, and/or artifacts in the first target region. Therefore, if the SNR of the first target region is lower than the first SNR threshold (i.e., doesn't satisfy the first preset condition), the processing device 140 may further analyze the first target region to determine the reason that causes the low SNR of the first target region. In some embodiments, when the one or more first parameter values don't satisfy the preset condition, the processing device 140 may determine whether the first target region includes lesions and/or image artifacts. For example, the processing device 140 may determine lesions in the first target region based on an abnormal point recognition technique disclosed in Chinese patent application Ser. No. 20/211,0983114.2, which is incorporated herein by reference. If the first target region includes no lesions and/or no image artifacts, the processing device 140 may determine that the target scan needs to be re-performed. If the first target region includes lesions and/or image artifacts, the processing device 140 may proceed to operation 1404.

In 1404, if the one or more first parameter values don't satisfy the preset condition, the processing device 140 (e.g., the determination module 220) may determine a second target region in the first image.

The second target region may provide reference information for comparing with the first target region. In some embodiments, the second target region may be different from the first target region. In some embodiments, the second target region may include a tissue region with uniform nuclide uptake. Exemplary second target regions may include a muscle, the brain, or the like, or any combination thereof. In some embodiments, the second target region may be determined according to clinical experience. For example, the first target region may be a liver region, and the second target region may include a region of the subject other than the liver region, such as a muscle region, a brain region, etc.

In some embodiments, the determination of the second target region in the first image may be similar to the determination of the first target region in the first image. For example, the processing device 140 may identify a second region (e.g., the muscle region, the brain region, etc.) from the second image, and then the processing device 140 may determine the second target region in the first image based on the second region. The second region may refer to a region representing one or more typical organs and/or tissues of the subject and correspond to the second target region in the second image.

In some embodiments, the processing device 140 may identify the second region in the second image through a machine learning model. An input of the machine learning model may be the second image, and an output of the machine learning model may be the second image that the second region is marked or a segmentation mask indicating the second region. In some embodiments, the machine learning model may be obtained by training a neural network model e.g., a graph neural network (GNN). For example, the machine learning model may be a trained neural network model, and stored in the imaging system 100 (e.g., the processing device 140, the storage device 150, etc.) through an interface. In some embodiments, the machine learning model may be a deep learning model. For example, the machine learning models may be obtained by training a 3D V-net or a 2.5D V-net segmentation model.

In some embodiments, the processing device 140 may identify the first region and the second region based on a same machine learning model. In some embodiments, the processing device 140 may identify the first region and the second region based on different machine learning models.

In some embodiments, the processing device 140 may determine the second target region in the first image based on the second region segmented from the second image. In some embodiments, the processing device 140 may determine the second target region in the first image by mapping the second region to the first image through a registration matrix. The registration matrix may be the same as or different from the registration matrix described in operation 1304.

In 1406, the processing device 140 (e.g., the determination module 220) may determine one or more second parameter values of the one or more quality parameters of the second target region.

The quality parameter may be used to represent the image quality. In some embodiments, the second parameter value(s) of the second target region may be determined in a similar manner to that of the first parameter value(s) of the first target region, which may not be repeated herein.

In 1408, the processing device 140 (e.g., the determination module 220) may determine, based on the one or more second parameter values, whether the target scan needs to be re-performed.

In some embodiments, the processing device 140 may determine whether the one or more second parameter values satisfy a second preset condition.

The second preset condition may include that a second parameter value of an SNR of the second target region exceeds or reaches a second SNR threshold, a second parameter value of a proportion of artifact regions in the second target region doesn't exceed a second proportion threshold, etc. In some embodiments, the second SNR threshold and/or the second proportion threshold may be determined based on system default setting (e.g., statistic information) or set manually by the user (e.g., a technician, a doctor, a physicist, etc.). For example, the second SNR threshold and/or the second proportion threshold may be input by a user through the terminal 130 and stored in the storage device 150. In some embodiments, the second SNR threshold may be the same as or different from the first SNR threshold. Similarly, the second proportion threshold may be the same as or different from the first proportion threshold. Correspondingly, the second preset condition may be the same as or different from the first preset condition.

If the one or more second parameter values satisfy the second preset condition, the processing device 140 may determine that the first target region includes the lesion and/or the image artifact. For instance, if the one or more second parameter values satisfy the second preset condition, the processing device 140 may determine the first image as an acceptable image. The processing device 140 may further determine that the reason why the one or more first parameter values of the first target region don't satisfy the first preset condition. For example, the reason may be that lesions and/or image artifacts exist in the first target region, which lowers the SNR of the first target region. In some embodiments, the processing device 140 may send a prompt to the user. For example, a text prompt “The first target region includes lesion/image artifact” may be displayed on a screen of the terminal 130.

If the one or more second parameter values don't satisfy the second preset condition, the processing device 140 may determine that the target scan needs to be re-performed. For example, if the one or more second parameter values don't satisfy the second preset condition, the processing device 140 may determine that the first image includes a large noise and is not acceptable. Further, the processing device 140 may determine that the target scan needs to be re-performed.

By using the one or more first parameter values and the one or more second parameter values to determine whether the target scan needs to be re-performed, whether the target scan needs to be re-performed may be determined based on a twice determination, which may reduce the likelihood of the determination error caused by once determination, thereby improving the accuracy of determining whether the target scan needs to be re-performed. In addition, the processing device 140 may further determine whether the first target region includes lesions and/or image artifacts and the reason why the one or more first parameter values of the first target region don't satisfy the first preset condition, which may not be determined by once determination. Therefore, the processing device 140 may determine whether the target scan needs to be re-performed based on the reason, which may improve the accuracy of determining whether the target scan needs to be re-performed and the efficiency of the target scan.

Merely by way of example, the liver region may be the first target region and the gluteus maximus region may be the second target region.

In some embodiments, the imaging device 110 may be a PET-CT device. The imaging device 110 may perform a CT scan on a subject from head to toe to obtain the second image of a whole body of the subject, and perform a PET scan on the subject from head to toe to obtain the first image of the whole body of the subject. The processing device 140 may first identify the liver region from the second image, and then use the registration matrix to map the liver region in the second image to the PET space to determine the liver region in the first image. Then, the processing device 140 may determine the one or more first parameter values of the one or more quality parameters of the liver region in the first image, and determine whether the one or more first parameter values satisfy the first preset condition. If the one or more first parameter values satisfy the first preset condition, the processing device 140 may determine that the first image of the liver region is acceptable.

In some embodiments, if the one or more first parameter values don't satisfy the first preset condition, the processing device 140 may determine that the first image of the liver region is not acceptable, and the target scan (i.e., the PET scan) may be re-performed.

In some embodiments, if the one or more first parameter values don't satisfy the first preset condition, the processing device 140 may further analyze the first image, for example, to determine whether the first image includes lesions and/or image artifacts in the liver region. Therefore, the processing device 140 may further obtain the gluteus maximus region in the first image. For example, the processing device 140 may first identify the gluteus maximus region from the second image, and then use the registration matrix to map the gluteus maximus region in the second image to the PET space to determine the gluteus maximus region in the first image.

Then, the processing device 140 may determine the one or more second parameter values of the one or more quality parameters of the gluteus maximus region in the first image, and determine whether the one or more second parameter values satisfy the second preset condition. If the one or more second parameter values don't satisfy the second preset condition, the processing device 140 may determine that the first image is not acceptable, and the target scan needs to be re-performed. If the one or more second parameter values satisfy the second preset condition, the processing device 140 may determine that the first image of the liver region is acceptable, and the liver region in the first image may include lesions and/or image artifacts. In some embodiments, the processing device 140 may send a prompt to prompt that the liver region may include lesions and/or image artifacts.

In some embodiments, the processing device 140 may determine whether the target scan needs to be re-performed after the first image of the whole body is obtained. In some embodiments, the processing device 140 may determine whether the target scan needs to be re-performed during the PET scan. For example, when the PET scan is performed from head to toe, the processing device 140 may determine whether the target scan needs to be re-performed after the liver region (or the gluteus maximus region) is scanned. The processing device 140 may send the prompt to the user based on a determination result, so that the user may adjust a scanning strategy in time.

In some embodiments, the first target region and the second target region may be interchangeable. For example, the first target region may be the gluteus maximus region, and the second target region may be the liver region. Further, the first region and the second region may be interchangeable, and the first preset condition and the second preset condition may be interchangeable.

It should be noted that the descriptions of the processes 400, 700, 800, 1300, and 1400 are provided for the purposes of illustration, and are not intended to limit the scope of the present disclosure. For persons having ordinary skills in the art, various variations and modifications may be conducted under the teaching of the present disclosure. For example, the processes 400, 700, 800, 1300, and 1400 may be accomplished with one or more additional operations not described, and/or without one or more of the operations discussed. Additionally, the order in which the operations of the processes 400, 700, 800, 1300, and 1400 is not intended to be limiting. However, those variations and modifications may not depart from the protection of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

What is claimed is:

1. A method for medical imaging, implemented on a computing device having at least one processor and at least one storage device, the method comprising:
obtaining a scout image of a subject lying on a scanning table, the scanning table including N portions corresponding to N bed positions of a target scan, and an i^{th} portion of the N portions corresponding to an i^{th} bed position of the N bed positions;
for the i^{th} bed position,
determining, based on the scout image, one or more body parts of the subject located at the i^{th} portion of the scanning table; and
determining at least one scanning parameter or reconstruction parameter corresponding to the i^{th} bed position based on the one or more body parts of the subject.

2. The method of claim 1, wherein the determining, based on the scout image, one or more body parts of the subject located at the i^{th} portion of the scanning table includes:
identifying, from the scout image, a plurality of feature points of the subject; and
determining, based on the plurality of feature points, the one or more body parts of the subject located at the i^{th} portion of the scanning table.

3. The method of claim 2, wherein the determining, based on the plurality of feature points, the one or more body parts of the subject located at the i^{th} portion of the scanning table includes:
obtaining a corresponding relationship between the plurality of feature points and a plurality of body part classifications;
determining, based on the scout image, a positional relationship between the plurality of feature points and the i^{th} portion of the scanning table; and
determining, based on the corresponding relationship and the positional relationship, the one or more body parts of the subject located at the i^{th} portion of the scanning table.

4. The method of claim 3, wherein the determining, based on the corresponding relationship and the positional relationship, the one or more body parts of the subject located at the i^{th} portion of the scanning table includes:
determining, based on the positional relationship, one or more target feature points located at the i^{th} portion of the scanning table;
for each of the plurality of body part classifications, determining, based on the corresponding relationship, a count of target feature points that belong to the body part classification; and
determining, based on the counts corresponding to the plurality of body part classifications, the one or more body parts of the subject located at the i^{th} portion of the scanning table.

5. The method of claim 3, wherein the determining, based on the corresponding relationship and the positional relationship, the one or more body parts of the subject located at the i^{th} portion of the scanning table includes:
determining, based on the positional relationship, one or more target feature points located at the i^{th} portion of the scanning table;
for each of the plurality of body part classifications, determining, based on the corresponding relationship, one or more key feature points that belong to the body part classification from the one or more target feature points; and
determining, based on the one or more key feature points corresponding to the one or more body part classifications, the one or more body parts of the subject located at the i^{th} portion of the scanning table.

6. The method of claim 1, wherein
the one or more body parts of the subject located at the i^{th} portion of the scanning table includes a body part having physiological motion, and
the at least one scanning parameter includes a motion detection parameter.

7. The method of claim 1, wherein the target scan is performed using a first imaging modality, and the method further includes:
obtaining a first image of the subject captured by the target scan and a second image of the subject captured by a reference scan, the reference scan being performed on the subject using a second imaging modality;
generating a third image based on the second image and an image prediction model, the third image being a predicted image of the subject corresponding to the first imaging modality; and
determining, based on the first image and the third image, whether the first image includes image artifacts.

8. The method of claim 7, wherein the first imaging modality is positron emission computed tomography (PET), and the second imaging modality is a computed tomography (CT) or a magnetic resonance (MR).

9. The method of claim 7, wherein in response to determining that the first image includes the artifact, the method further comprises:
obtaining a plurality of candidate image blocks of the first image by moving a sliding window on the first image;
for each of the plurality of candidate image blocks, determining a similarity degree between the candidate image block and a corresponding image block in the third image; and
determining, based on the similarity degrees of the plurality of candidate image blocks, one or more target image blocks as one or more artifact regions of the first image.

10. The method of claim 9, further comprising:
generating, based on the first image and the one or more target image blocks, one or more incomplete images; and
generating a corrected first image based on the one or more incomplete images and an image recovery model.

11. The method of claim 1, further comprising:
obtaining a first image of the subject captured by the target scan;
determining a first target region in the first image;
determining one or more first parameter values of one or more quality parameters of the first target region; and
determining, based on the one or more first parameter values, whether the target scan needs to be re-performed.

12. The method of claim 11, wherein the determining, based on the one or more first parameter values, whether the target scan needs to be re-performed includes:
determining whether the one or more first parameter values satisfy a preset condition;
in response to determining that the one or more first parameter values satisfy the preset condition, determining a second target region in the first image;
determining one or more second parameter values of the one or more quality parameters of the second target region; and
determining, based on the one or more second parameter values, whether the target scan needs to be re-performed.

13. The method of claim 11, wherein the first target region is a liver region, and the second target region includes a region of the subject other than the liver region.

14. The method of claim 11, wherein the one or more quality parameters include at least one of a signal noise ratio (SNR) or a proportion of artifact regions in the first target region or the second target region.

15. A system for medical imaging, implemented on a computing device having at least one processor and at least one storage device, the method comprising:
at least one storage device including a set of instructions; and
at least one processor configured to communicate with the at least one storage device, wherein when executing the set of instructions, the at least one processor is configured to direct the system to perform operations including:
obtaining a scout image of a subject lying on a scanning table, the scanning table including N portions corresponding to N bed positions of a target scan, and an i^{th} portion of the N portions corresponding to an i^{th} bed position of the N bed positions;
for the i^{th} bed position,
determining, based on the scout image, one or more body parts of the subject located at the i^{th} portion of the scanning table; and
determining at least one scanning parameter or reconstruction parameter corresponding to the i^{th} bed position based on the one or more body parts of the subject.

16. The system of claim 15, wherein the determining, based on the scout image, one or more body parts of the subject located at the i^{th} portion of the scanning table includes:
identifying, from the scout image, a plurality of feature points of the subject; and
determining, based on the plurality of feature points, the one or more body parts of the subject located at the i^{th} portion of the scanning table.

17. The system of claim 15, wherein
the one or more body parts of the subject located at the i^{th} portion of the scanning table includes a body part having physiological motion, and
the at least one scanning parameter includes a motion detection parameter.

18. The system of claim 15, wherein the target scan is performed using a first imaging modality, and the operations further include:
obtaining a first image of the subject captured by the target scan and a second image of the subject captured by a reference scan, the reference scan being performed on the subject using a second imaging modality;
generating a third image based on the second image and an image prediction model, the third image being a predicted image of the subject corresponding to the first imaging modality; and
determining, based on the first image and the third image, whether the first image includes image artifacts.

19. The system of claim 15, further comprising:
obtaining a first image of the subject captured by the target scan;
determining a first target region in the first image;
determining one or more first parameter values of one or more quality parameters of the first target region; and
determining, based on the one or more first parameter values, whether the target scan needs to be re-performed.

20. A non-transitory computer readable medium, comprising executable instructions that, when executed by at least one processor, direct the at least one processor to perform a method, the method comprising:
obtaining a scout image of a subject lying on a scanning table, the scanning table including N portions corresponding to N bed positions of a target scan, and an i^{th} portion of the N portions corresponding to an i^{th} bed position of the N bed positions;
for the i^{th} bed position,
determining, based on the scout image, one or more body parts of the subject located at the i^{th} portion of the scanning table; and
determining at least one scanning parameter or reconstruction parameter corresponding to the i^{th} bed position based on the one or more body parts of the subject.
